(19) United States
(12) Patent Application Publication
(10) Pub. No.: US 2012/0022048 A1
(43) Pub. Date: Jan. 26, 2012

(54) Anti-proliferative substituted pyrazolo[3,4-d]pyrimidines derivatives (SPP) to inhibit immune activation, virus replication and tumor growth

(76) Inventors: Franco Lori, Bethesda, MD (US); Davide De Forni, (US); Michael Ray Stevens, (US)

(21) Appl. No.: 12/804,551

(22) Filed: Jul. 22, 2010

(51) Int. Cl. A61K 31/519; A61K 31/55; A61P 37/00; A61P 31/12; A61P 35/02; A61K 31/5377; A61P 35/00

(52) U.S. Cl. 514/217.06; 514/262.1; 514/234.2

(57) ABSTRACT

A family of pyrazolo[3,4-d]pyrimidine derivatives (SPPs) with different substituents on the pyrimidine and pyrazolo rings have been characterized with a panel of tests demonstrating their effects in cell proliferation, toxicity, apoptosis and inhibition of virus replication. We have identified compounds and molecular structures suitable for the treatment of viral infection because they have antiviral activity, anti-proliferative activity or, preferably, both so that, as a single molecule, they both limit T cell hyperactivation and inhibit virus replication. These compounds are not toxic at effective concentrations and are poorly apoptotic. Other nontoxic compounds within this family with excellent anti-proliferative and apoptotic features are potentially effective as anti-cancer drugs.

### FIELD OF THE INVENTION

The present invention relates to the treatment of chronic viral and bacterial infections and/or cancer by the use of selected pyrazolo[3,4-d]pyrimidine derivatives with different kinds of substitutes on the pyrimidine ring and pyrazolo ring (SPP). We found that certain members of the SPP family of compounds inhibit T cell hyperactivation or hyper-proliferation, a pathogenetic factor in infectious and neoplastic diseases, but do not interfere with antigen-specific immune responses. That is, they inhibit hyperactivation without generating undesired immune suppression or immune deficiency. Within the family we have also identified compounds with potent antiviral and potent anticancer activities using a novel test system.

### BACKGROUND OF INVENTION

Pyrazolo[3,4-d]pyrimidine derivatives are a class of compounds generally known to include members that might be useful as anticancer drugs. Some of them have been tested for Abl and Src tyrosine kinase inhibition. Some were active against both, some only against Abl and some only against Src. Si121 was not tested (Carraro, F., et al., Pyrazolo[3,4-d]pyrimidines as potent anti-proliferative and proapoptotic agents toward A431 and 8701-BC cells in culture via inhibition of c-Src phosphorylation. J Med Chem 2006, 49, 1549-1561 and Manetti, F., et al., Inhibition of Bcr-Abl phosphorylation and induction of apoptosis by pyrazolo[3,4-d]pyrimidines in human leukemia cells. ChemMedChem 2007, 2 343-353).

U.S. patent application Ser. No. 10/558,553 filed Aug. 14, 2006 by Bondavalli discloses and claims Pyrazolo[3,4-d]pyrimidine derivatives and their potential use in cancer treatment. There is no disclosure of discussion of cellular proliferation or anti-viral activity.

Synthesis of 1-(2-chloro-2-phenylethyl)-6-methylthio-1H-pyrazolo[3,4-d]pyrimidines 4-amino substituted and their biological evaluation, Schenone, et al., European Journal of Medicinal Chemistry Volume 39, Issue 2, February 2004, Pages 153-160 discloses that a new series of 4-amino-6-methylthio-1H-pyrazolo[3,4-d]pyrimidines bearing the 2-chloro-2-phenylethyl chain at the N1 position, has been synthesized. The affinity of these compounds for A1 adenosine receptor (A1AR) was measured. The compounds showed poor affinity. A more interesting result was obtained by some compounds, which demonstrated anticancer activity in a human epidermoid carcinoma cell line. This experiment involved a single cancer cell line A431, but not primary cells.

U.S. Pat. No. 5,981,533 issued to Traxler et al. Nov. 9, 1999 discloses 4-amino-1H-pyrazolo[3,4-d]pyrimidine derivatives and intermediates for their manufacture. The compounds are said to inhibit especially the tyrosine kinase activity of the receptor for epidermal growth factor and can be used, for example, in the case of epidermal hyperproliferation (psoriasis) and as anti-tumor agents.

U.S. Pat. No. 5,977,086 issued to Lisziewicz and Lori Nov. 2, 1999 discloses that hydroxyurea (HU) has been widely used in cancer therapy as a broad-spectrum antineoplastic drug, and that it is useful in combination with other drugs for the treatment of HIV infection.

U.S. Pat. No. 6,046,175 issued Apr. 4, 2000 to Lori et al specifies that HU is best associated with didanosine (ddI) to exert antiviral activity, in addition to the intrinsic anti-hyperactivation and anti-proliferation properties of HU itself.

WO/2008/079460 re “TYROSINE KINASE INHIBITORS FOR PREVENTION OR TREATMENT OF INFECTION” discloses the use of kinase inhibitors for the treatment of acute pathogenic infections, including among others HIV infection. These initiators include inhibitors of tyrosine kinase, preferably, Abelson (Abl) and/or Src-family tyrosine kinases, or pharmaceutically acceptable salts, enantiomers, analogs, esters, amides, prodrugs, metabolites, or derivatives thereof. In Table A, amongst others Imatinib is presented, under the code name: cGP-2-STI571. The above invention does not disclose the SPPs.

WO 2006044968 re “COMBINATION THERAPY FOR TREATING VIRAL INFECTIONS” discloses a method of treating viral infections, particularly Hepatitis B (HBV) and Human Immunodeficiency Virus (HIV) infections, by administering elvucitabine and a second active agent to a patient suffering viral infection. The second active agent is, for example, an immunomodulatory compound, an anti-viral agent, or a combination comprising one or more of the foregoing active agents. For example the anti-viral agent may be a tyrosine kinase inhibitor, a CCR5 inhibitor, a non-nucleoside reverse transcriptase inhibitor, a protease inhibitor, an integrase inhibitor. The patent further discloses that Tyrosine kinase inhibitors have been found to have antiviral properties and may be used as the second active agent. Tyrosine kinases inhibitors included in this invention were Imatinib mesylate (GLEEVEC® or GLIVEC®, Novartis), Gefitinib (IRESSA®, Astra Zeneca), and erlotinib (TARCEVA®, OSI Pharmaceuticals). Several other tyrosine kinase inhibitors are mentioned. For example, AMNI07 (Tasigna™ from Novartis Pharma AG) which is now approved for use in the USA and the EU for drug-resistant chronic myelogenous leukemia (CML) and sunitinib malate (also known as SUI 1248, brand name SUTENT®, from Pfizer) which is now being marketed for the treatment of gastrointestinal and advanced renal cell carcinoma. Combinations of elvucitabine and sunitinib malate and optionally AMNI07 are said to be within the scope of the invention. The above invention does not disclose SPP.

WO2005117885A1 re “Imanitib for treating liver disease and viral infections” discloses the use of imanitib for treating hepatitis C, influenza virus, rhinovirus, etc. The above invention does not disclose the SPPs.

Other recently approved TKIs include NEXAVAR (sorafenib tosylate), which is marketed by Bayer and Bayer Schering Pharma for both liver and renal cell carcinoma, and TYKERB (lapatinib ditosylate), marketed by GSK for breast cancer.

### SUMMARY OF THE INVENTION

We have discovered that compounds having the following formula are particularly useful because they are able to both limit viral replication and T cell hyperactivation.

4-substituted derivatives of pyrazolo[3,4-d]pyrimidine (SPPs) that inhibit cell proliferation and viral replication have the formula

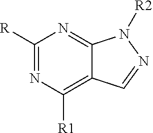

wherein:
R=SC2H5, or an alkylthio group
R1=NHC4H9, NHCH2CH2C6H5, NHCH2C6H5, NHC6H4-mCl, NHCH2C6H4-pF, NHC3H7, NHCH2CH2C6H4-mCl, 4-MORPHOLINYL, NHCH2C6H4-oCl

### R2=CH2CHClC6H5, CH2CHClC6H4-pF, CH2CHClC6H4-pCl

Pyrazolo[3,4-d]pyrimidine derivatives with a 2-chloro-2-phenylethyl or a 2-chloro-2-(4-fluoro-phenyl)ethyl or a 2-chloro-2-(4-chloro-phenyl)ethyl side chain at N1 (substituent R2) are particularly preferred to inhibit cell proliferation and/or viral replication.

Pyrazolo[3,4-d]pyrimidine derivatives with a SC2H5 or alkylthio side chain at C6 (substituent R) are particularly preferred to inhibit cell proliferation and/or viral replication.

An advantage of these compounds is that they have multiple utilities, that is, as a single compound they are antiviral, or have the ability to limit viral replication, they are anti-proliferative, that is, they limit T cell hyperproliferation, and yet they have low cellular toxicity, that is, they are NOT pro-apoptotic.

Compounds that are particularly preferred as AV-HALTS are designated herein as VS1-002, VS1-003, VS1-004, VS1-008, VS1-024, VS1-026, VS1-027, VS1-028, VS1-029, VS1-031, VS1-036.

We have discovered that the following compounds are useful in that they limit cellular proliferation.

4-substituted derivatives of pyrazolo[3,4-d]pyrimidine (SPPs) that inhibit cellular proliferation have the formula

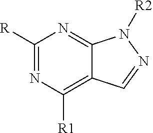

wherein:

### R=SC2H5, SCH3, H, N(CH3)2

R1=NHC4H9, NHCH2CH2C6H5, NHCH2C6H5, NHC6H4-mCl, NHCH2C6H4-pF, NHC3H7, NHCH2CH2C6H4-mCl, 4-MORPHOLINYL, N(C2H5)2, NH(CH2)2OC2H5, 1-HEXAHYDROAZEPINYL, NHCH2C6H4-pCl, NHCH2C6H4-oCl
R2=CH2CHClC6H5, CH2CHClC6H4-pF, CH2CHClC6H4-pCl,

Such compounds can be useful for treatment of tumors and leukemia, particularly if they also tend to induce apoptosis.

We have discovered that the following compounds are useful in that they limit viral replication:

4-substituted derivatives of pyrazolo[3,4-d]pyrimidine (SPPs) that inhibit viral replication have the formula

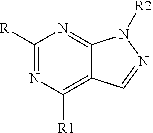

### Wherein:

### R=H, SCH3, NH(CH2)2OH

### R1=1-HEXAHYDROAZEPINYL, NHCH2CH2C6H5,4-MORPHOLINYL, NHCYCLOHEXYL, NHCH2C6H5, NHC6H4-mCl

### R2=CHCHC6H5

Compounds that are classified as antiviral activity have the following designations in this application: VS1-001, VS1-005, VS1-012, VS1-015, VS1-016

We have discovered also a group of compounds with potential use as anti-cancer compounds. These are antiviral or anti-proliferative, and PRO-apoptotic.

4-substituted derivatives of pyrazolo[3,4-d]pyrimidine (SPPs) useful to induce apoptosis have the formula

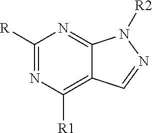

wherein:

### R=SC2H5, SCH3, H, SCH2CH2-4-MORPHOLINYL

R1=NHCH2CH2C6H5, NHCH2C6H5, NHC6H4-mCl, 1-HEXAHYDROAZEPINYL, NHC3H7, 4-MORPHOLINYL, NHCH2C6H4-pCl

### R2==CH2CHClC6H5, CH2CHBrC6H5, CH2CHClC6H4-pF, CH2CHClC6H4-pCl

In addition to being effective for limiting viral replication and/or limiting T cell replication, these compounds also induce apoptosis, and so are particularly useful for treating tumors and leukemia as opposed to a chronic viral infection such as HIV infection.

Compounds particularly preferred for cancer treatment have the following designations in this application: VS1-010, VS1-011, VS1-020, VS1-022, VS1-023, VS1-030, VS1-032, VS1-033, VS1-034, VS1-035.

Another way to view the utility of all of these compounds is that they are all useful to limit cell cycle progression beyond the G1/S checkpoint.

The advantages of the present inventions can be obtained by administering an effective amount of a compound having the desired properties to cells in order to obtain the desired results. The compounds may be formulated in a pharmaceutical composition comprising the compound, or a physiologically acceptable salt thereof, having the desired effect, and one or more suitable excipients. Suitable excipients are well known in the art.

### BRIEF DESCRIPTION OF THE DRAWINGS

FIG. 1 shows the chemical structures for different compounds that could be candidates for use as antiviral, anticancer or antiviral-hyperactivation limiting therapeutics (AV-HALTS).

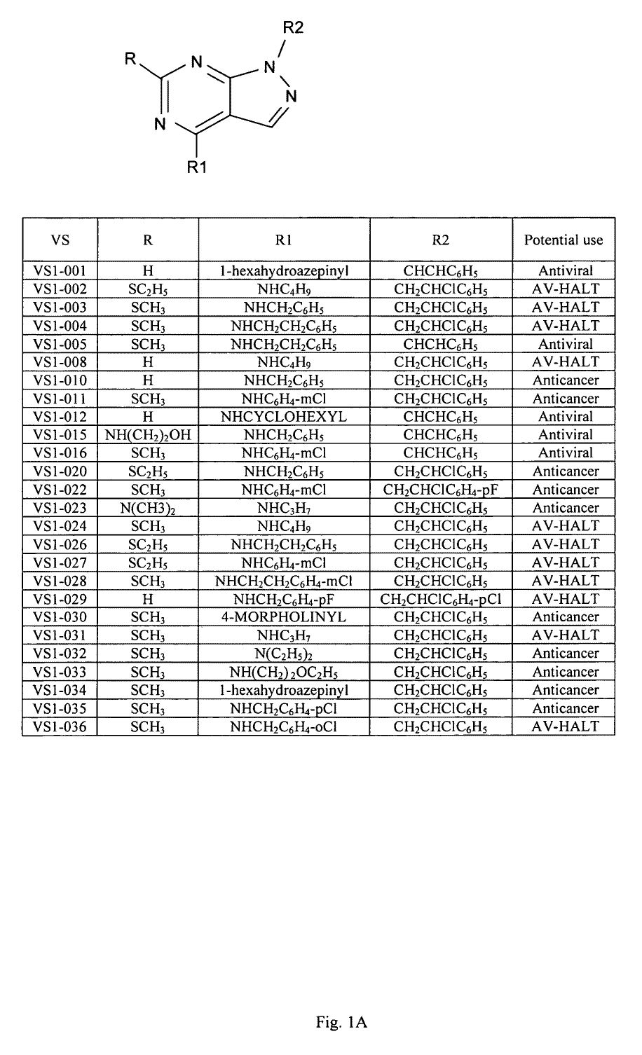

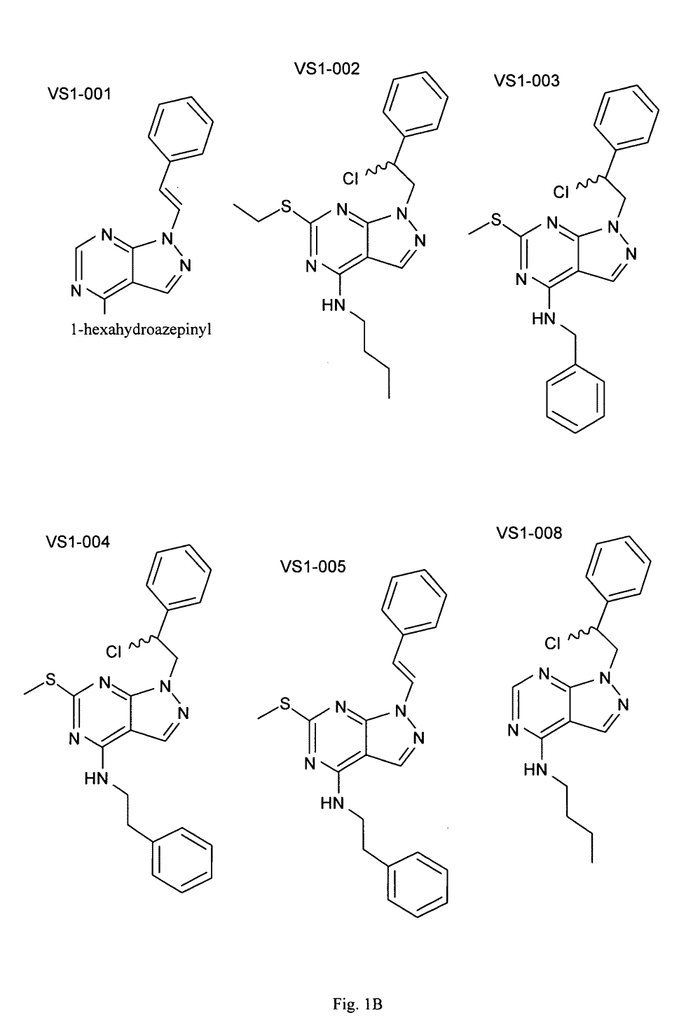

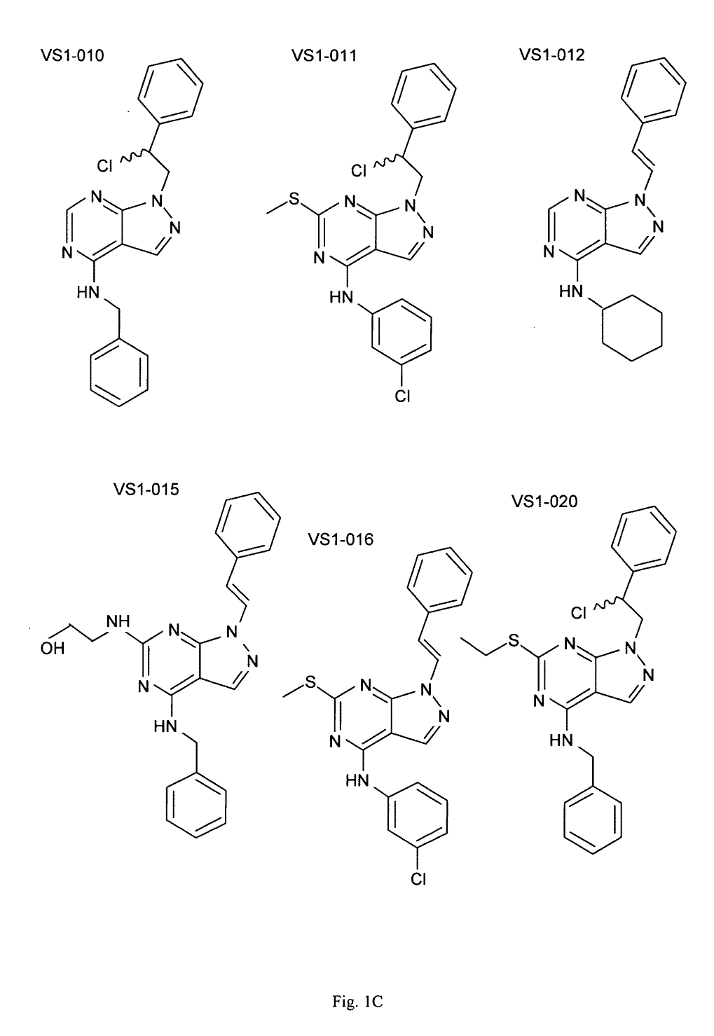

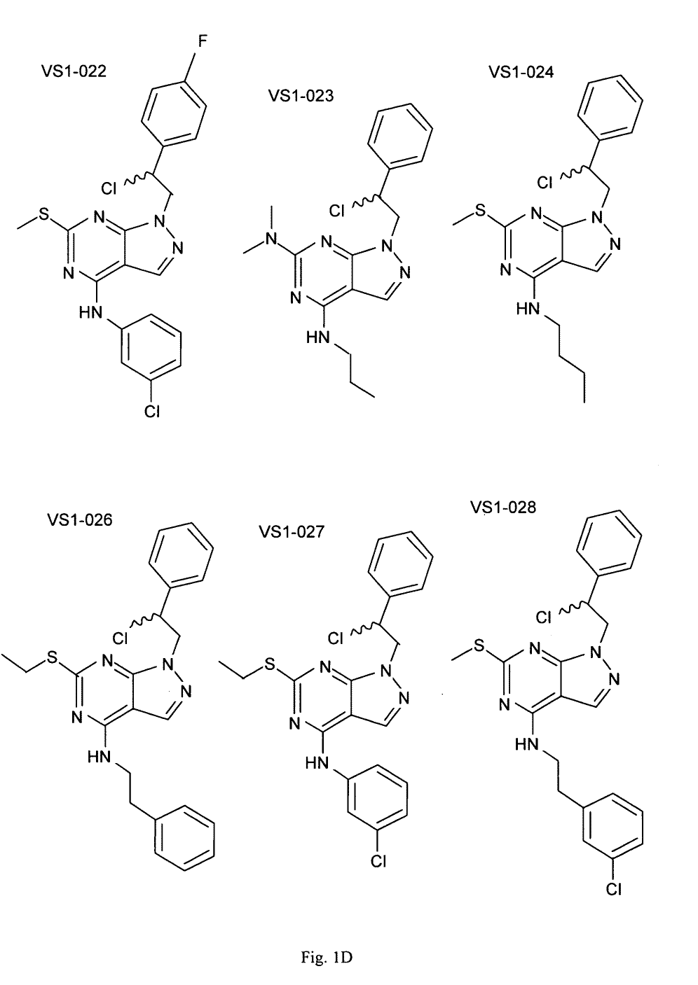

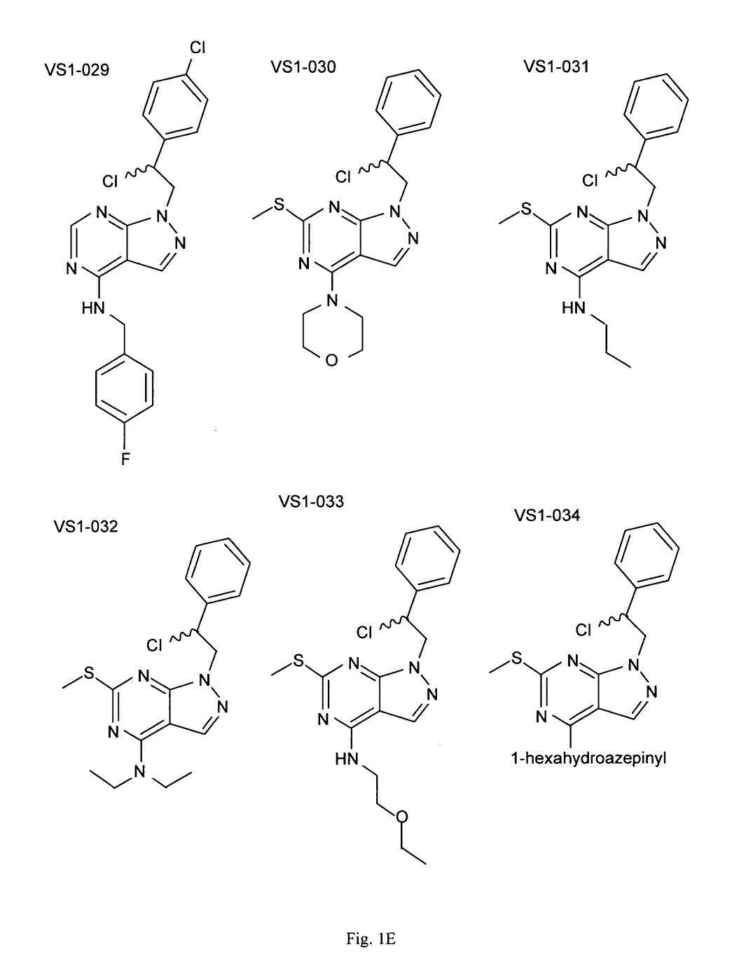

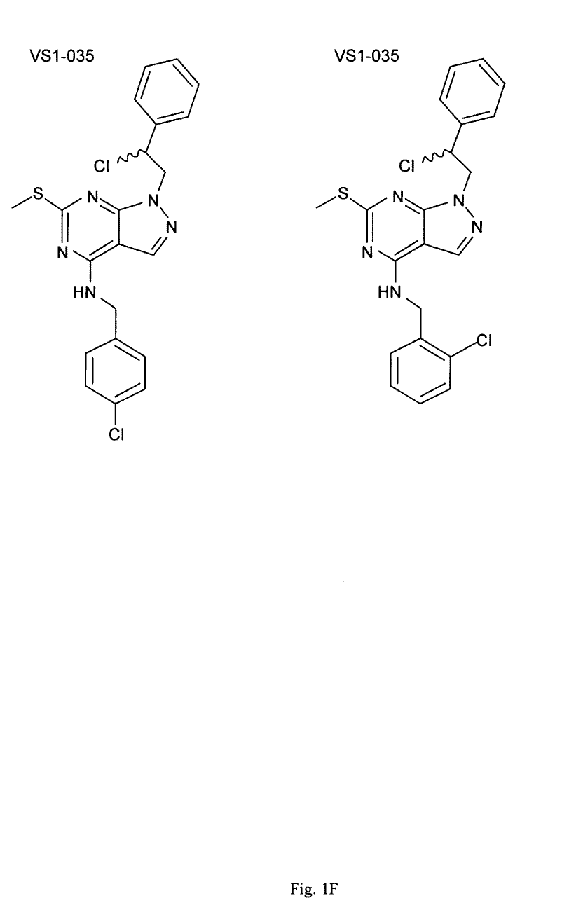

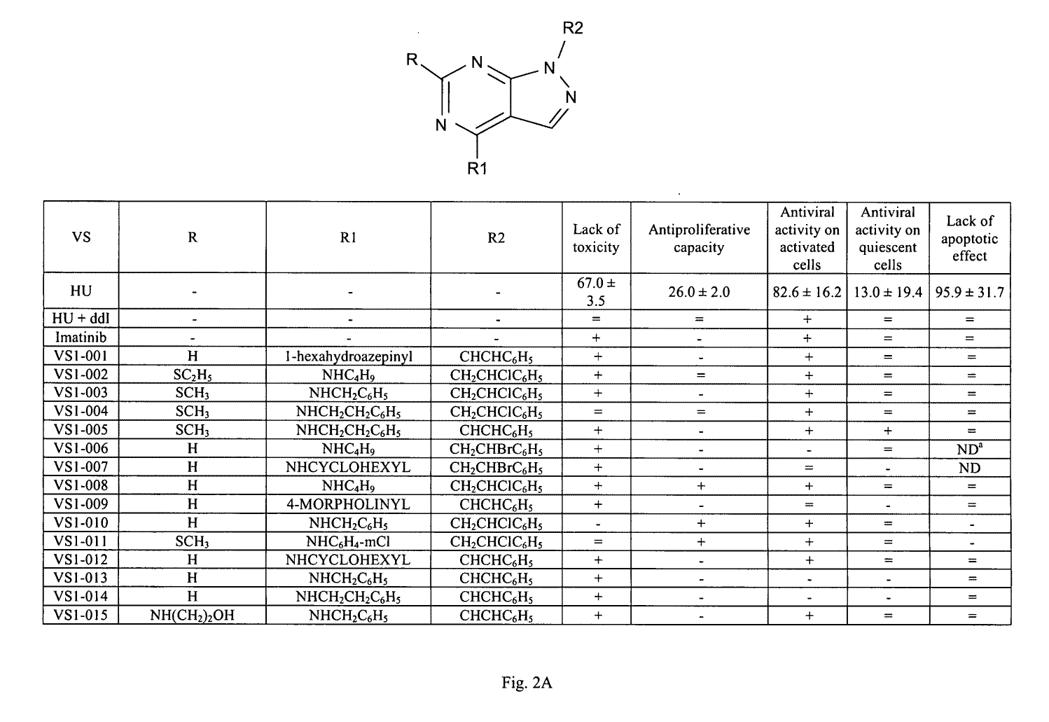

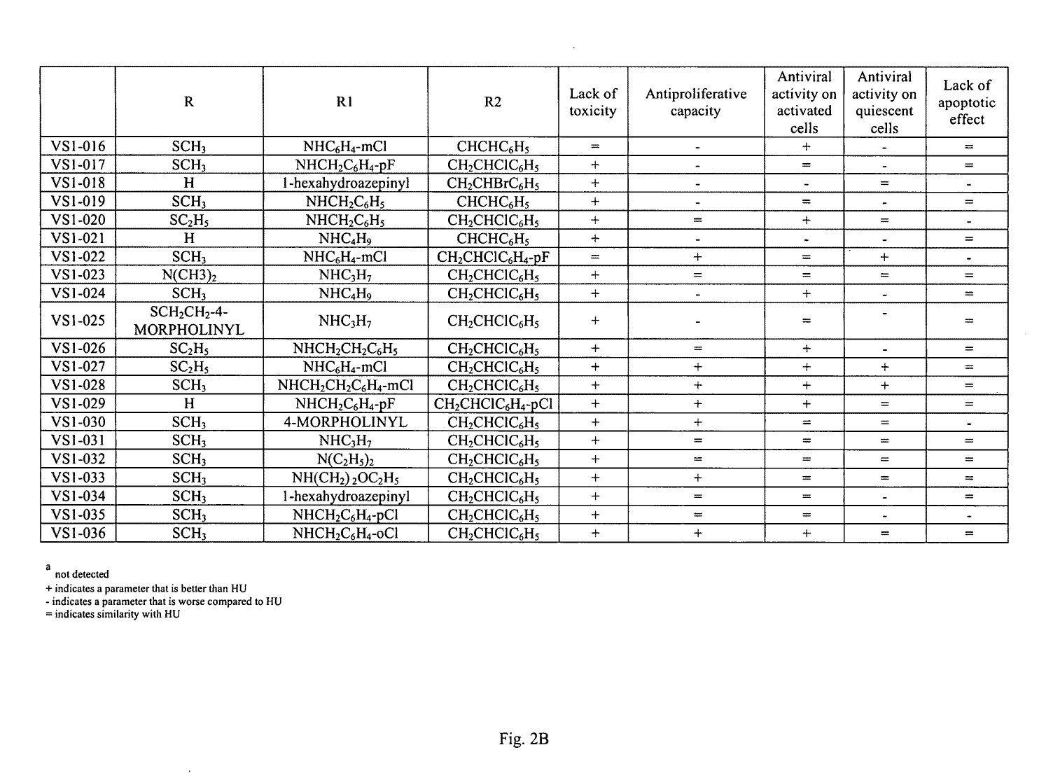

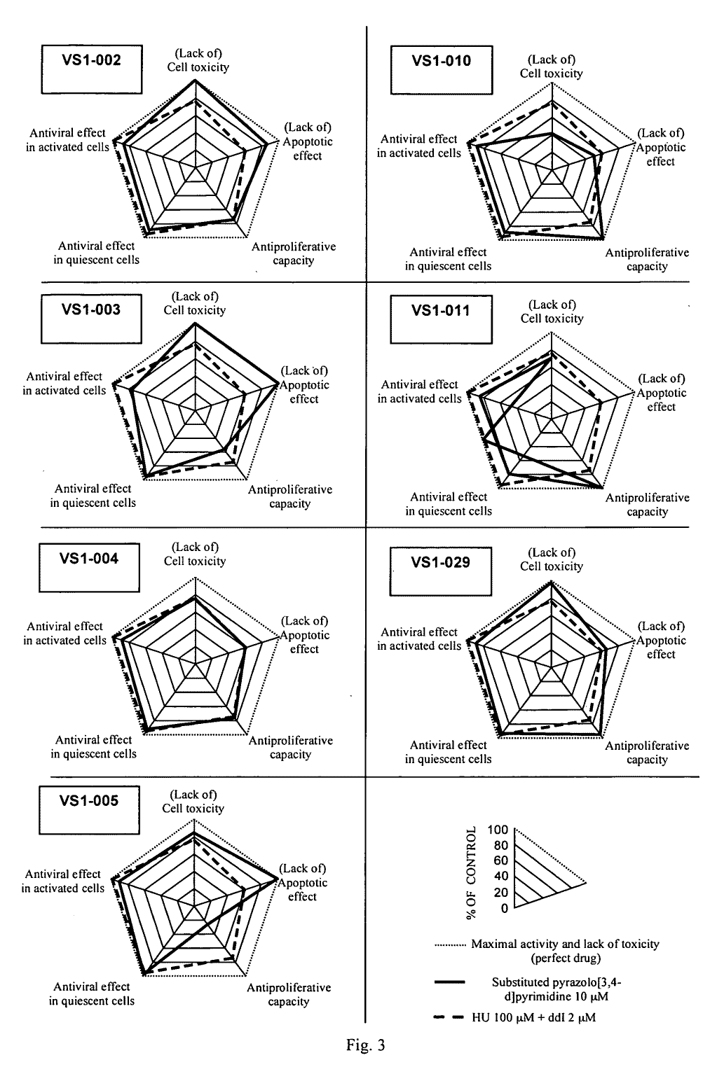

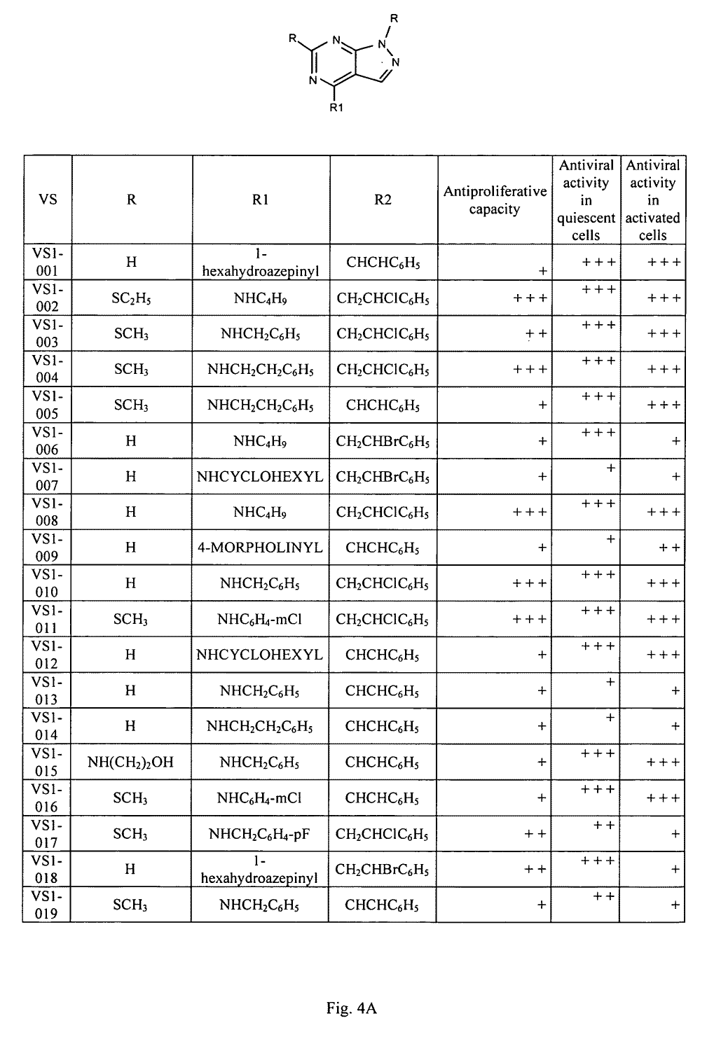

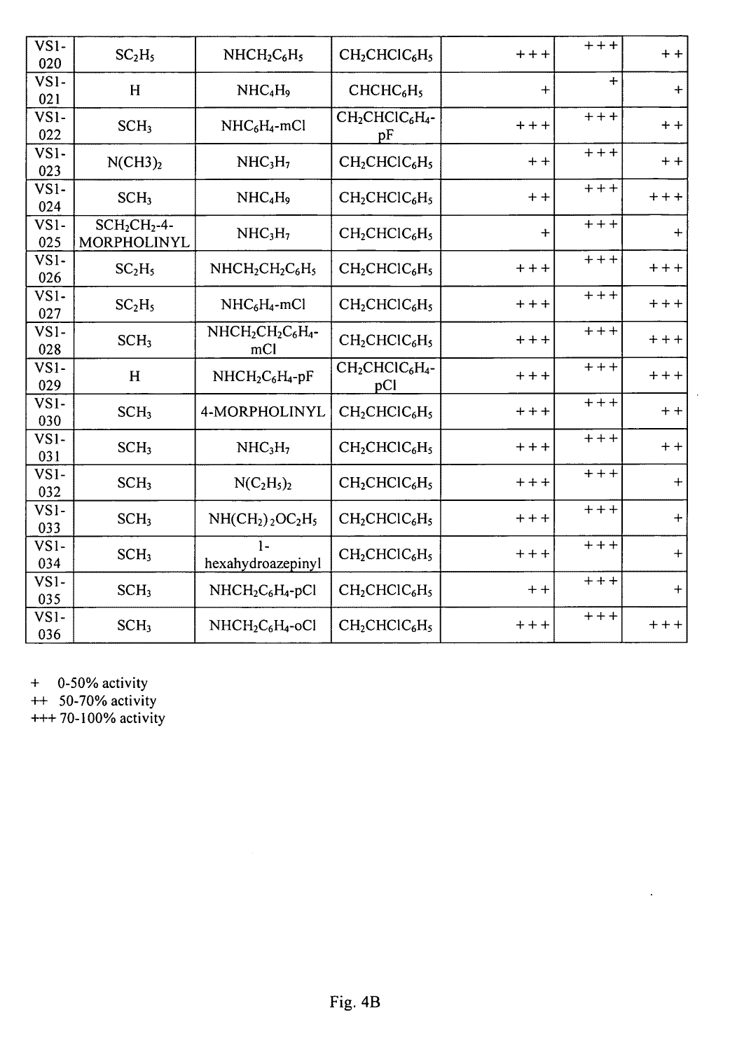

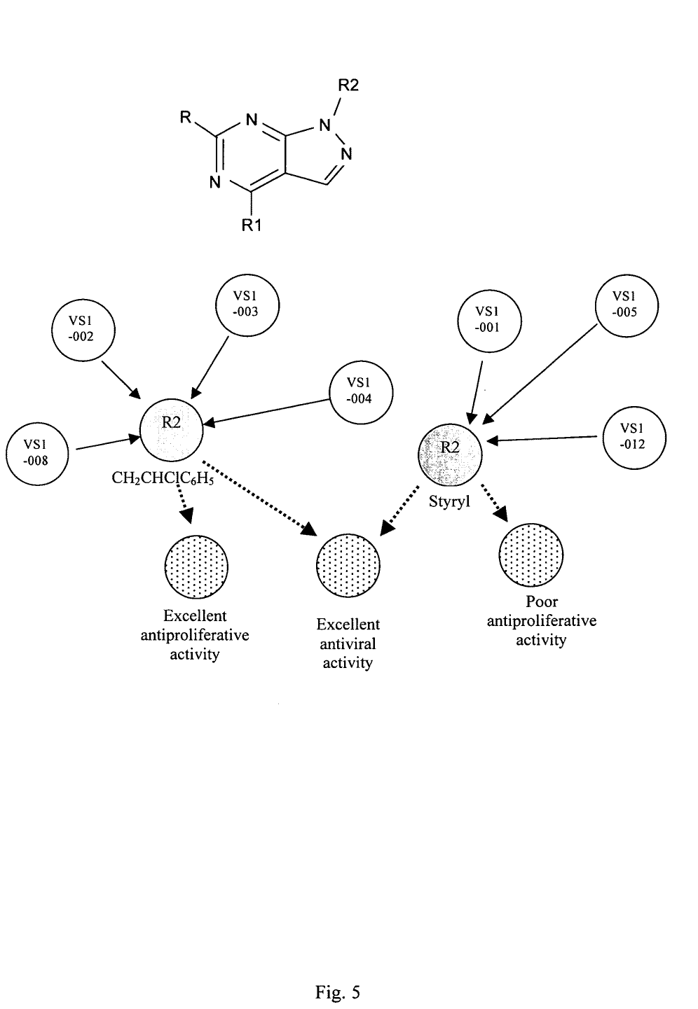

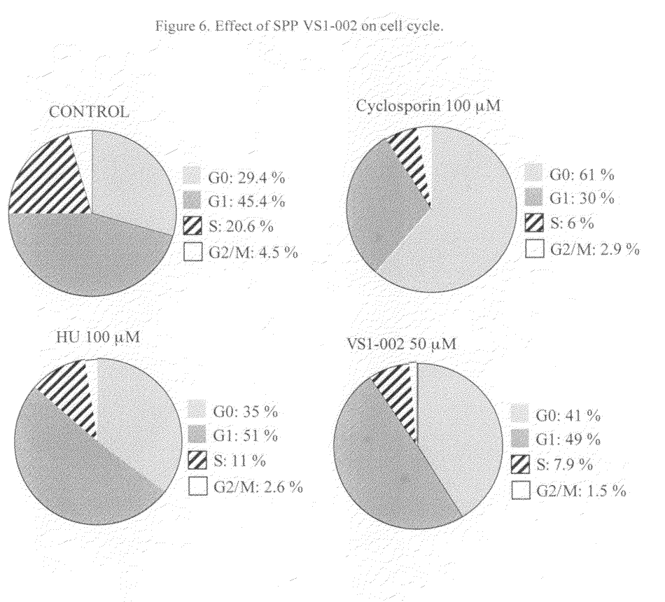

VS1-002 or N-butyl-1-(2-chloro-2-phenylethyl)-6-(ethylthio)-1H-pyrazolo[3,4-d]pyrimidin-4-amine;

VS1-003 or 1-(2-chloro-2-phenylethyl)-6-(methylthio)-N-benzyl-1H-pyrazolo[3,4-d]pyrimidin-4-amine

VS1-004 or 1-(2-chloro-2-phenylethyl)-6-(methylthio)-N-(2-phenylethyl)-1H-pyrazolo[3,4-d]pyrimidin-4-amine;

VS1-005 or 1-styryl-6-methylthio-N-(2-phenylethyl)-1H-pyrazolo[3,4-d]pyrimidin-4-amine

VS1-010 or N-benzyl-1-(2-chloro-2-phenylethyl)-1H-pyrazolo[3,4-d]pyrimidin-4-amine;

VS1-011 or N-(3-chlorophenyl)-1-(2-chloro-2-phenylethyl)-6-(methylthio)-1H-pyrazolo[3,4-d]pyrimidin-4-amine;

VS1-029 or 1-[2-chloro-2-(4-chlorophenyl)ethyl]-N-(4-fluorobenzyl)-1H-pyrazolo[3,4-d]pyrimidin-4-amine.

FIG. 2 is a single table of experimental results showing relative (lack of) toxicity (expressed as viability of cells compared to untreated control), anti-proliferative activity on primary blood cells (proliferation is expressed through the percentage mitotic index compared to untreated control), anti-HIV activity in activated and quiescent cells (HIV replication is expressed as the percentage amount of p24 production compared to untreated control), and (lack of) apoptotic effects (percentage of apoptotic effects compared to untreated control), if any, for 36 compounds compared to HU. “+” indicates a parameter that is better than HU, “−” indicates a parameter that is worse compared to HU. “=” indicates similarity with HU. It has to be noted that HU was employed at 100 μM while all SPPs were tested at 10 μM, that means that 10 times less SPP has the same effect, or even higher than HU.

FIG. 3 is a set of “Spider diagrams” for seven different compounds, that is, a way of visualizing the way a drug meets a group of five desired parameters (lack of toxicity, lack of apoptotic effect, anti-proliferative capacity and antiviral effect in activated and quiescent cells). A “perfect” drug would meet 100% of all parameters, and in this case, would make a perfect pentagon at 100% (outer dotted line of the graph). The combination of HU and ddI is used as a comparator.

FIG. 4 shows the relationships between substituents of the SPP and activity towards cell proliferation and viral replication. “+++” indicates excellent activity, “++” good activity and “+” poor activity.

FIG. 5 shows an example from the model used to find the relationships between the substituents and the activity of the SPPs.

FIG. 6 shows the effect of a specific compound, VSI-002, on the cell cycle.

### DETAILED DESCRIPTION OF THE INVENTION

### Protein tyrosine kinases

A protein kinase is an enzyme that modifies other proteins chemically. They covalently attach a phosphate group to the side chain of tyrosine, serine, or threonine residues found on proteins. The human genome contains about 500 protein kinase genes. The family of enzymes known as protein tyrosine kinases (PTKs) act on tyrosine, and provide an essential role in the normal regulation of cell growth. Various protein tyrosine kinases (TK) catalyze the phosphorylation of specific tyrosyl residues in various proteins involved in the regulation of cell growth and differentiation. Phosphorylation often results in a functional change to the target protein, or substrate. PTKs are involved in the transduction and processing of extracellular and intracellular signals. They play a critical role in regulating normal cell growth and differentiation, and if they malfunction, they are also known to play a role in oncogenesis. That is, inappropriate or uncontrolled activation of many of these kinases, i.e., aberrant protein tyrosine kinase activity, for example by over-expression or mutation, has been shown to result in uncontrolled cell growth.

U.S. Pat. No. 7,507,741 discloses that protein kinases have been implicated as targets in central nervous system disorders such as Alzheimer's, pain sensation, inflammatory disorders such as arthritis, bone diseases such as osteoporosis, cancer, atherosclerosis, thrombosis, metabolic disorders such as diabetes, blood vessel proliferative disorders such as angiogenesis, restenosis, autoimmune diseases and transplant rejection and infectious diseases such as viral and fungal infections.

Aberrant activity of PTKs has been implicated in human malignancies, such as non-small cell lung, bladder and head and neck cancers, breast, ovarian, gastric and pancreatic cancers.

Aberrant PTK activity has also been implicated in a variety of other disorders: psoriasis, fibrosis, atherosclerosis, restenosis, autoimmune disease, allergy, asthma, transplantation rejection inflammation, thrombosis, bronchitis, and nervous system diseases. Specific tyrosine kinases have been implicated in disease conditions in which T cells are hyperactive e.g., rheumatoid arthritis, autoimmune disease, allergy, asthma, and graft rejection. The process of angiogenesis has been associated with a number of disease states (e.g., tumorogenesis, psoriasis, rheumatoid arthritis) and this has been shown to be controlled through the action of a number of receptor PTKs.

There exist two classes of PTKs: the receptor tyrosine kinases and the cytoplasmic or receptor-associated tyrosine kinases. The former have a transmembrane receptor with a tyrosine kinase domain protruding into the cytoplasm. The transmembrane receptor is an extracellular ligand binding domain and the tyrosine kinase domain is an intracellular catalytic domain with intrinsic tyrosine kinase activity. Receptor tyrosine kinases play an important role in regulating cell division, cellular differentiation, and morphogenesis (development of shape, typically of embryos.)

Receptor-associated tyrosine kinases are tyrosine kinases recruited from the cytoplasm to a receptor following hormone binding, and are involved in a number of signaling cascades, principally those involved in cytokine signaling (but also others, including growth hormone). One such receptor-associated tyrosine kinase is Janus kinase (JAK), many of whose effects are mediated by STAT proteins. Receptor-associated tyrosine kinases transmit signals from the membrane through the interaction with the cytoplasmic domain of membrane proteins. Activated Tyrosine kinases produce a variety of downstream effects that ultimately result in changes in gene expression.

During the past years, much effort has been made to elucidate the structure and function of cytoplasmic PTKs, particularly of c-Src tyrosine kinases, because they are active in several tumors, including breast, bone, colon, lung, pancreatic, ovarian, head and neck, bladder, neuronal cancers as well as in chronic myelogenous leukemia and multiple myeloma. Abl tyrosine kinase is another cytoplasmic PTK. It is involved in the development of chronic myeloid leukemia (CML). Several tyrosine kinase inhibitors (TKIs) have been approved as anticancer agents, such as dasatinib and Imatinib for the treatment of CML, as well as gefitinib and erlotinib for the treatment of non-small-cell lung cancer. Many other TKIs are in development in clinical trials and they are a growing class of anticancer agents.

Some experimental SPPs have been tested in vitro as dual Src/Abl inhibitors in several tumor cell lines. These SPPs have different substituents on the pyrimidine and pyrazolo rings. Such compounds possess the ability to interfere with the phosphorylation activity of both Src and Abl and, consequently, to show anti-cancer activity in cell lines (Carraro, F., et al., Pyrazolo[3,4-d]pyrimidines as potent anti-proliferative and proapoptotic agents toward A431 and 8701-BC cells in culture via inhibition of c-Src phosphorylation. J Med Chem 2006, 49, 1549-1561).

The effects of the experimental SPPs were assessed on leukemia cell lines in order to evaluate their activity on proliferation and apoptosis, or programmed cell death. (1-(tert-butyl)-3-(4-chlorophenyl)-4-aminopyrazolo-[3,4-d]pyrimidine (PP2), a potent inhibitor of both Src and Abl tyrosine kinases, was used as the reference standard. SPPs showed a significant anti-proliferative activity on K-562 cells, with an IC₅₀ spanning from 19 to 176 micromolar. The compounds were also tested in MEG-01 cells with activity values similar to those obtained with the other cell lines. To confirm in each cell line the existence of a direct link between anti-proliferative activity and inhibition of the tyrosine kinases, the phosphorylation of the TK (in this case a variant of Abl, “Bcr-Abl” and Src) and its downstream substrate (STAT-5) was evaluated. A reduction in phosphorylation of all targets was revealed, strongly suggesting that the effects mediated by the compounds on proliferation of leukemia cells are a consequence of the reduction of the kinase activity. Some of the compounds were also evaluated for their proapoptotic activity on a Poly-ADP-Ribose-Polymerase (PARP) assay. The compounds induced apoptosis of leukemia cells to different extents. In summary, several compounds were characterized by a biological profile comparable to, or better than, that found for the reference compound PP2, in some cases showing a submicromolar inhibitory activity toward the enzyme and a significant anti-proliferative activity toward three leukemia cell lines (Manetti, F., et al., Inhibition of Bcr-Abl phosphorylation and induction of apoptosis by pyrazolo[3,4-d]pyrimidines in human leukemia cells. ChemMedChem 2007, 2 343-353).

### Normal Cell Cycle

The cell cycle, or cell-division cycle, is the series of events that take place in a cell leading to its division and duplication (proliferation). In cells without a nucleus (prokaryotes), the cell cycle occurs via a process termed binary fission. In cells with a nucleus (eukaryotes), the cell cycle can be divided in two brief periods: interphase—during which the cell grows, accumulating nutrients needed for mitosis and duplicating its DNA—and the mitosis (M) phase, during which the cell splits itself into two distinct cells, often called “daughter cells.” The cell-division cycle is a vital process by which a single-celled fertilized egg develops into a mature organism, as well as the process by which hair, skin, blood cells, and some internal organs are renewed.

The cell cycle consists of four distinct phases: G1 phase, S phase (synthesis), G2 phase (collectively known as interphase) and M phase (mitosis). M phase is itself composed of two tightly coupled processes: mitosis, in which the cell's chromosomes are divided between the two daughter cells, and cytokinesis, in which the cell's cytoplasm divides forming distinct cells. Activation of each phase is dependent on the proper progression and completion of the previous one. Cells that have temporarily or reversibly stopped dividing are said to have entered a state of quiescence called G0 phase.

After cell division, each of the daughter cells begins the interphase of a new cycle. Although the various stages of interphase are not usually morphologically distinguishable, each phase of the cell cycle has a distinct set of specialized biochemical processes that prepare the cell for initiation of cell division.

The term “post-mitotic” is sometimes used to refer to both quiescent and senescent cells. Nonproliferative cells in multicellular eukaryotes generally enter the quiescent G0 state and may remain quiescent for long periods of time, possibly indefinitely (as is often the case for neurons). This is very common for cells that are fully differentiated. Cellular senescence is a state that occurs in response to DNA damage or degradation that would make a cell's progeny nonviable; it is often a biochemical alternative to the self-destruction of such a damaged cell by apoptosis.

The first phase within interphase, from the end of the previous M phase until the beginning of DNA synthesis is called G1 (G indicating gap). During this phase the biosynthetic activities of the cell, which had been considerably slowed down during M phase, resume at a high rate. This phase is marked by synthesis of various enzymes that are required in S phase, mainly those needed for DNA replication. Duration of G1 is highly variable, even among different cells of the same species. The ensuing S phase starts when DNA synthesis commences; when it is complete, all of the chromosomes have been replicated, i.e., each chromosome has two (sister) chromatids. Thus, during this phase, the amount of DNA in the cell has effectively doubled, though the number of chromosomes in the cell remains the same. Rates of RNA transcription and protein synthesis are very low during this phase. An exception to this is histone production, most of which occurs during the S phase. The duration of S phase is relatively constant among cells of the same species.

The cell then enters the G2 phase, which lasts until the cell enters mitosis. Again, significant protein synthesis occurs during this phase, mainly involving the production of microtubules, which are required during the process of mitosis. Inhibition of protein synthesis during G2 phase prevents the cell from undergoing mitosis.

The relatively brief M phase consists of nuclear division (karyokinesis) and cytoplasmic division (cytokinesis). In plants and algae, cytokinesis is accompanied by the formation of a new cell wall. The M phase has been broken down into several distinct phases, sequentially known as prophase, prometaphase, metaphase, anaphase, and telophase leading to cytokinesis.

Regulation of the cell cycle involves processes crucial to the survival of a cell, including the detection and repair of genetic damage as well as the prevention of uncontrolled cell division. The molecular events that control the cell cycle are ordered and directional; that is, each process occurs in a sequential fashion and it is impossible to “reverse” the cycle.

It is possible, however, to prevent progression of cell cycle and hold the cell cycle at certain phases. Since most of HIV expression occurs during the S Phase (Foli, A., et al., A Checkpoint in the Cell Cycle Progression as a Therapeutic Target to Inhibit HIV Replication. J Infect Dis, 2007. 196(9): p. 1409-15), it would be desirable to develop molecules that prevent cells from progressing beyond the G1/S checkpoint, like HU.

### T Cell Hyperactivation/Hyperproliferation

The condition where T cells engage in uncontrolled cell division is called T cell hyperactivation or hyperproliferation. T cells are immune system cells that can develop the capacity to kill infected or neoplastic cells. When T cells are contacted by antigens they become activated, or sensitized, and proliferate, that is, appear in greater numbers. This is a normal physiological process, which is useful to protect the host from the “sick cells” (tumor cells and infected cells).

However, excessive T cell activation, and particularly prolonged excessive activation, called T cell hyperactivation or -hyperproliferation, can contribute to disease progression and is considered a key pathogenetic factor in several chronic diseases such as cancer, and prolonged infectious diseases including HIV/AIDS infection. HIV infects T cells and depends on actively dividing or proliferating cells to serve as a means of replication. The infected cells produce HIV particles, which is an antigen. Antigenic stimulation by HIV particles, in turn, sustains further T cell activation or proliferation, as mentioned before. In addition to direct antigenic stimulation by HIV, microbial translocation across impaired gut-associated lymphoid tissues (GALT) throughout the course of HIV disease also sustains elevated T cell activation/proliferation (Brenchley, J. M., et al., Microbial translocation is a cause of systemic immune activation in chronic HIV infection. Nat Med, 2006. 12(12): p. 1365-71). This chronic cycle of events, over time, exhausts the immune system. Therefore, limiting cell T cell hyperactivation and hyperproliferation will suppress HIV replication, limit the loss of functional CD4 T helper cells and slow disease progression.

### Cancer and the Leukemia Model

Abnormal cell proliferation is necessary for leukemogenesis and the increase in tumor cell number accounts for the disease. The goal of current therapies is to decrease the number of tumor cells. Therefore, we have used a hyperactivated primary T cell model to study the anticancer activity of the compounds and determined the cytostatic and apoptotic effects of various compounds on hyperproliferating cells.

To validate our primary cell model we chose the reference compounds hydroxyurea (HU) and a tyrosine kinase inhibitor (Imatinib), as both have been used for the treatment of chronic myeloid leukemia (CML). Historically, Imatinib has substituted HU as first line treatment of CML. We have tested for other unexpected similarities in inhibiting cell growth (i.e. proliferation, apoptosis). In addition, we have some SPPs that possess antiviral activity similar to that of currently used antiviral compounds.

SPPs Compared with the Anticancer and Antiviral Drug Hydroxyurea

Hydroxyurea (HU) is an anti-proliferative agent indicated to treat different neoplastic as well as non-neoplastic diseases such as sickle cell anemia and psoriasis. HU has been used for the treatment of HIV infected individuals, especially in combination with antiretroviral drugs, such as ddI. HU inhibits the cellular enzyme ribonucleotide reductase, thus blocking the transformation of ribonucleotides into deoxyribonucleotides, depleting the intracellular deoxynucleotide triphosphate (dNTP) pool, and arresting the cell cycle in the G1/S phase (Lori, F., Hydroxyurea and HIV: 5 years later—from antiviral to immune-modulating effects. Aids, 1999. 13(12): p. 1433-42). HU strongly inhibits viral deoxyribonucleic acid (DNA) synthesis and has synergistic anti-HIV activity when combined with nucleoside reverse transcriptase inhibitors (NRTIs). Clinical trials have shown that HU-containing regimens effective in patients with varying degrees of treatment experience at different stages of infection.

HU can suppress virus replication by slowing down the rate of T cell proliferation (because HIV-1 needs actively dividing cells to replicate). To create a more effective antiviral drug that limits both HIV replication and immune hyperactivation (unwanted proliferation of the immune system), we wanted to identify a single compound containing all the positive aspects of HU and ddI combination. The present invention discloses such novel antiviral/anti-proliferative compounds (AV-HALTS) from the family of SPPs designated VS1-002, VS1-003, VS1-004, VS1-005, VS1-010, VS1-011, VS1-029, VS1-031, VS1-036 that meet all the following characteristics.
1. Compounds that have anti-proliferative activity, because (a) inhibiting cell proliferation leads to inhibition of HIV replication and (b) T cell hyperactivation or hyperproliferation is a key pathogenic factor in immune diseases.
2. Compounds that are active in quiescent cells, similar to hydroxyurea
3. Compounds that are active in activated T cells, representing an improvement over HU.
4. Compounds sharing both antiviral and anti-hyperactivation or anti-proliferation activities, an improvement over the two drug combination of HU and ddI.
5. Compounds that have low cellular toxicity at effective dose levels.
6. Compounds that have no or low apoptotic activity
   We have established new methodologies for in vitro screening and pre-clinical development of novel drugs to control HIV replication. First, we determine the toxicity of a given compound by testing several concentrations in primary T cells (as one of the major target of toxicities in vivo) and we select a concentration that corresponds to a viability >40% compared to an untreated control. We set this threshold because concentrations of drugs used in vivo generally correspond to an in vitro viability greater or equal to 40%. The selected concentration is then tested for anti-proliferative effect, apoptotic effect. To test the antiviral effect, we set up two systems in quiescent and in activated primary T cells. There are drugs such as HU that inhibit HIV replication only in quiescent cells, while antiviral drugs such as didanosine can inhibit HIV replication also in activated cells, because antivirals directly inhibit viral enzymes required for virus replication. Therefore, if a compound works in the activated cells system it has a direct anti-HIV mechanism of action. By using such an experimental system we found significant differences in activities, and could distinguish among potential anti-cancer drugs (low toxicity, potent proapoptotic and potent anti-proliferative), antiviral drugs (low toxicity, potent antiviral, absence of anti-proliferative capacity and not inducing apoptosis) and AV-HALTs (low toxicity, potent antiviral, potent anti-proliferative and not inducing apoptosis). The ability to distinguish among ineffective, antiviral, anticancer and AV-HALT molecules is a new and surprising result.

The results are tabulated in FIG. 2. At 10 μM of each compound showed an acceptable toxicity profile (viability above 40% compared to untreated control). Results regarding the induction of apoptosis varied among the compounds (sign “−” in FIG. 2 represents proapoptotic compounds because the column shows “lack of apoptosis”). During our investigations, we surprisingly found that all of the compounds have different characteristics: some compounds had neither antiviral nor anti-proliferative activity, some had good antiviral activity only, some had good anti-proliferative activity, some induced apoptosis, and some not. Most surprisingly, we found that many SPPs at 10 μM had the same or even higher anti-proliferative and antiviral potency than HU at 100 μM, suggesting 10 fold higher potency.

HU at 100 μM concentration was incorporated in all experimental studies as a comparator (FIG. 2). This concentration was not toxic, that is, the viability of the cells treated with this compound at this concentration was above 40 percent. HU 100 μM did substantially decrease cell proliferation but, HU alone was not effective in inhibiting HIV replication in activated T cells. However, very importantly, potent antiviral activity of HU has been demonstrated on quiescent cells, which are one of the major reservoirs of HIV. HU did not substantially affect the percentage of total apoptotic events, a feature that is useful in antiviral compounds. In contrast, many known potent anticancer compounds operate by inhibiting cell proliferation and inducing apoptosis.

The experiments on toxicity, anti-proliferative capacity, apoptosis, antiviral effect on both activated and quiescent cells gave the following results for the various tested SPPs, which are tabulated in FIG. 2:

VS1-001 did not have toxicity, was less effective than HU in decreasing cell proliferation; however, more importantly, it was a very effective inhibitor of HIV replication in both activated and quiescent T cells and similarly to HU, did not induce apoptosis. This compound is best characterized as an antiviral drug.”

VS1-002 10 μM was not toxic, had an anti-proliferative effect comparable to that of 100 μM HU and superior to that of Imatinib. This compound has anti-proliferative activity and antiviral activity in both quiescent and in hyperactivated cells. Differently from 100 μM HU, even at 10 μM it inhibited HIV replication in activated T cells. The compound inhibited the production of HIV-1 indirectly by reducing cellular proliferation (the majority of HIV-1 replication occurs in actively dividing cells) and also had a direct anti-HIV activity. It was not proapoptotic. This compound is an antiviral drug with anti-proliferative capacity, and we define it as an AV-HALT (AntiViral-Hyperactivation Limiting Therapeutic) drug.

VS1-003 was about as effective as VS1-002 in inhibiting HIV, with anti-proliferative capacity somewhat less than that of VS1-002. It did not induce apoptosis. This compound is an AV-HALT.

VS1-004 10 μM was not toxic, had good antiviral potency in both activated and quiescent cells, was as anti-proliferative as 100 μM HU, and only induced a small apoptotic effect. This compound is classified as an AV-HALT.

VS1-005 was not toxic and had anti-proliferative activity but slightly lower compared to HU. It had a strong anti-HIV activity in quiescent cells. The effect on apoptosis of VS1-005 was similar to, and perhaps slightly better than, that of HU, meaning it did not induce apoptosis. Unlike HU, this compound was active also against HIV in activated T cells. This compound is characterized as an antiviral drug.

VS1-006 was not effective either against proliferation or HIV in activated T cells.

VS1-007 at a concentration of 10 μM was the weakest compound tested to date. It showed poor performance against cell proliferation and it had no effect on HIV replication in activated T cells.

VS1-008 had no toxicity, showed good activity against proliferation (even better than HU though 10 times less concentrated) and HIV in both activated and quiescent cells. It did not induce apoptosis. This compound is an AV-HALT.

VS1-009 had very little activity with a profile similar to VS1-006 and VS1-007.

VS1-010 and VS1-011 10 μM had acceptable toxicity and both had increased anti-proliferative capacity compared to 100 μM HU and were active as antivirals in both activated and quiescent cells. They induced apoptosis. The combination of anti-proliferative, antiviral, and apoptosis-inducing qualities suggests that these compounds will be potent anticancer drugs.

VS1-012 through VS1-016 showed poor or no anti-proliferative activity. Nonetheless, VS1-012, VS1-015 and VS1-016 showed better anti-HIV activity than HU. These compounds are characterized as antiviral drugs.

VS1-017, VS1-018, VS1-019, VS1-021, and VS1-025 had poor activity both versus proliferation and HIV.

VS1-020 and VS1-022 had no toxicity, were as anti-proliferative or even better than HU. They were effective against HIV either in both quiescent and activated T cells. They were both proapoptotic and can be suggested as anticancer drugs.

VS1-023 was not toxic, showed anti-proliferative and antiviral activity similar to HU though it was 10 times less concentrated. It was not proapoptotic. Because it is similar to HU in our system but less toxic, it could be suitable to replace HU as an anti-hyperproliferation and anticancer drug.

VS1-024 and VS1-026 showed a good toxicity profile, were equal to or a little less anti-proliferative than HU and slightly less potent against HIV. They were not proapoptotic. These compounds are AV-HALTs.

VS1-027, VS1-028 and VS1-029 10 μM showed a good toxicity profile, good anti-proliferative capacity (superior to 100 μM HU) and good anti-HIV activity in both activated and quiescent cells. They were no more apoptotic than HU. These compounds are AV-HALTs.

VS1-030 had a good toxicity profile, at 10 μM it was greater than 100 μM HU and 10 μM Imatinib in inhibiting cell proliferation, and it was proapoptotic. This is a potentially potent anticancer drug.

VS1-032, VS1-033 and VS1-034 had anti-proliferative activity comparable or even greater than HU. They could be anticancer drugs.

VS1-035 had good anti-proliferative activity, similar to HU, and proapoptotic activity. The toxicity profile was good. This compound is a potential anticancer drug.

VS1-031 and VS1-036 were not toxic, at 10 μM had anti-proliferative capacity greater or equal to that of 100 μM HU, were effective against HIV in activated and quiescent cells. They did not induce apoptosis. These compounds are AV-HALTs.

Results are shown graphically in FIG. 3 for seven representative compounds. Each spider graph shows the results for five parameters measured for the compound (10 μM), in comparison with the combination of HU (100 μM)+ddI (2 μM). Each arm of the graph corresponds to a different parameter: lack of cell toxicity (100%=maximal viability of the cells, that is the same viability of the non treated control), lack of apoptotic effect (100%=no induction of apoptosis compared to not treated control), anti-proliferative capacity (100%=complete inhibition of cell proliferation compared to not treated control), antiviral effect in activated and quiescent cells (100%=complete inhibition of HIV replication compared to not treated control). A “perfect” drug would meet 100% of all parameters, and would make a perfect pentagon at 100% (outer dotted line of the graph). SPPs are represented with a solid line, HU+ddI combination is represented with a dashed line. Surprisingly, VS1-002 at 10 μM has the same activity of HU 100 μM+ddI 2 μM combination, 20% less toxicity and more than 20% less proapoptotic effect. VS1-003 has an activity profile similar to HU+ddI (it is slightly less potent against proliferation and HIV in activated cells), with less toxicity and no effect on apoptosis. VS1-004 is as effective as HU+ddI in inhibiting cell proliferation and HIV, with the same level of toxicity and apoptosis. These three compounds show a graph comparable to that of HU+ddI and can be classified as AV-HALTs. VS1-005 has the same toxicity of HU+ddI, no effect on apoptosis, has the same antiviral power but lacks anti-proliferative capacity (50% less than HU+ddI). This compound has a profile of a purely antiviral drug. VS1-010 has a good activity against HIV, is 20% more anti-proliferative than HU+ddI, it is more toxic and more proapoptotic compared to the two drug combination. VS1-011 has activity comparable to that of HU+ddI but it is extremely proapoptotic, as shown graphically by the fact that the point on the “lack of apoptotic effect” arm is on the negative scale. These two compounds have a profile of anticancer drugs. VS1-029 is again similar to HU+ddI, even more anti-proliferative (about 20% more), and is another AV-HALT candidate.

We established that the presence of a 2-chloro-2-phenylethyl (bearing or not halogen substituents in the phenyl ring) side chain at the N1 position (R2) or of SC2H5 group at C6 position of pyrazolo[3,4-d]pyrimidine derivatives was strongly associated with anti-proliferative activities. The presence of either a 2-bromo-2-phenylethyl side chain or a styryl group at the N1 position (R2) of pyrazolo[3,4-d]pyrimidine derivatives was negatively associated with anti-proliferative activity. The presence of either a 2-chloro-2-phenylethyl side chain at the N1 position, a styryl side chain at the position N1 and a SCH₃ or SC2H5 group at the position C6 (R), a NHC₆H₄-mCl group at position C4 (R1) of pyrazolo[3,4-d]pyrimidine derivatives were independently from the other groups were associated with antiviral activity. These compounds were active against HIV on activated T cells, representing an improvement over HU (FIG. 2, a “+” indicates that antiviral activity is greater than that of HU). The presence of a 2-bromo-2-phenylethyl side chain at the N1 position (R2) of pyrazolo[3,4-d]pyrimidine derivatives was negatively associated with antiviral activity.

Structure-activity relationships are shown in FIG. 4. “+” indicates 0 to 50% activity (poor activity), “++” indicates 50 to 70% activity (good activity), “+++” indicates more than 70% activity (excellent activity), compared to not treated control. Compounds with a 2-chloro-2-phenylethyl side chain in R2 position or an alkylthio group in position R are always associated with good (++) or excellent (+++) anti-proliferative or antiviral activity. FIG. 4 shows that the presence of either a 2-bromo-2-phenylethyl side chain or a styryl group at the R2 position often corresponds to poor anti-proliferative capacity (only one +). The presence of a styryl side chain at the position R2 and a SCH₃ or SC2H5 group at the position R, a NHC₆R₄-mCl group at position R1 of pyrazolo[3,4-d]pyrimidine derivatives were independently associated with good (++) or excellent (+++) antiviral activity.

In FIG. 5 we show how we could define the relations between structure and activity of the compounds. We built up a matrix and used a computer model that correlates all the substituents with the compounds that bear those substituents and with poor, good or excellent (lack of) toxicity, anti-proliferative capacity, antiviral activity, (lack of) apoptotic effect, the same parameters we used for the spidergraphs. Correlations are shown as arrows. This figure shows one example of the correlations one can find by using this model. The compounds bearing a 2-chloro-2-phenylethyl group have both excellent anti-proliferative and antiviral activity while compounds bearing a styryl group possess only antiviral activity.

FIG. 6 illustrates another way of viewing the properties of a compound. The following Examples further describe but do not limit the inventions described herein, or the claims that follow.

### EXAMPLES

### Example 1

### Study of the Toxicity of the SPPs

We investigated whether the selected compounds have a toxicity profile (in terms of cell viability) comparable to that of HU (at the dosage commonly used in vivo Cmax=100 μM) and to that of Imatinib (tested at 10 μM like the other SPPs).

First, these drugs were tested in activated T cells to determine the highest non-toxic concentration, that is, the concentration where cell viability was more than 40% compared to the control. Human peripheral blood mononuclear cells (PBMC), obtained from healthy, normal donors, were stimulated for two days with PHA 5 mg/ml. IL-2 was then added (20 U/ml), cells were plated (10⁵ cells per well in 200 ml) and drug exposure was begun. Different concentrations of the compounds were tested in triplicate with HU employed as a comparator. After 7 days of incubation at 37° C., cells were stained with Trypan Blue and counted using a hemocytometer. The number of dead and living cells was counted at the microscope. The percentage of living cells over the total number of cells was calculated. Viability was expressed as percentage of live cells compared to the untreated control, indicated as 100%.

### Toxicity of SPPs

TABLE 1
Viability of the cells was measured after treatment with SPPs for seven consecutive
days. Viability was expressed as a percentage of the not treated control (NT), indicated as 100%.

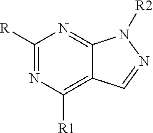

| VS | R | R1 | R2 | % viability |
|---|---|---|---|---|
| HU | — | — | — | 67.0 ± 3.5 |
| HU + ddI | — | — | — | 75.9 ± 10.8 |
| Imatinib | — | — | — | 105.0 ± 7.0 |
| VS1-001 | H | 1-hexahydroazepinyl | CHCHC₆H₅ | 136.3 ± 26.8 |
| VS1-002 | SC₂H₅ | NHC₄H₉ | CH₂CHClC₆H₅ | 108.9 ± 8.7 |
| VS1-003 | SCH₃ | NHCH₂C₆H₅ | CH₂CHClC₆H₅ | 103.4 ± 4.0 |
| VS1-004 | SCH₃ | NHCH₂CH₂C₆H₅ | CH₂CHClC₆H₅ | 74.9 ± 13.8 |
| VS1-005 | SCH₃ | NHCH₂CH₂C₆H₅ | CHCHC₆H₅ | 84.3 ± 22.7 |
| VS1-006 | H | NHC₄H₉ | CH₂CHBrC₆H₅ | 142.1 ± 56.8 |
| VS1-007 | H | NHCYCLOHEXYL | CH₂CHBrC₆H₅ | 88.3 ± 13.4 |
| VS1-008 | H | NHC₄H₉ | CH₂CHClC₆H₅ | 111.3 ± 45.3 |
| VS1-009 | H | 4-MORPHOLINYL | CHCHC₆H₅ | 91.4 ± 28.8 |
| VS1-010 | H | NHCH₂C₆H₅ | CH₂CHClC₆H₅ | 40.9 ± 35.8 |
| VS1-011 | SCH₃ | NHC₆H₄-mCl | CH₂CHClC₆H₅ | 70.4 ± 16.9 |
| VS1-012 | H | NHCYCLOHEXYL | CHCHC₆H₅ | 117.7 ± 39.1 |
| VS1-013 | H | NHCH₂C₆H₅ | CHCHC₆H₅ | 92.1 ± 23.2 |
| VS1-014 | H | NHCH₂CH₂C₆H₅ | CHCHC₆H₅ | 81.4 ± 15.2 |
| VS1-015 | NH(CH₂)₂OH | NHCH₂C₆H₅ | CHCHC₆H₅ | 82.0 ± 5.8 |
| VS1-016 | SCH₃ | NHC₆H₄-mCl | CHCHC₆H₅ | 71.2 ± 13.0 |
| VS1-017 | SCH₃ | NHCH₂C₆H₄-pF | CH₂CHClC₆H₅ | 90.3 ± 7.6 |
| VS1-018 | H | 1-hexahydroazepinyl | CH₂CHBrC₆H₅ | 96.0 ± 5.2 |
| VS1-019 | SCH₃ | NHCH₂C₆H₅ | CHCHC₆H₅ | 90.9 ± 3.2 |
| VS1-020 | SC₂H₅ | NHCH₂C₆H₅ | CH₂CHClC₆H₅ | 90.6 ± 2.7 |
| VS1-021 | H | NHC₄H₉ | CHCHC₆H₅ | 92.6 ± 6.9 |
| VS1-022 | SCH₃ | NHC₆H₄-mCl | CH₂CHClC₆H₄-pF | 76.4 ± 14.9 |
| VS1-023 | N(CH3)₂ | NHC₃H₇ | CH₂CHClC₆H₅ | 85.9 ± 20.5 |
| VS1-024 | SCH₃ | NHC₄H₉ | CH₂CHClC₆H₅ | 84.6 ± 11.0 |
| VS1-025 | SCH₂CH₂-4-MORPHOLINYL | NHC₃H₇ | CH₂CHClC₆H₅ | 93.3 ± 16.0 |
| VS1-026 | SC₂H₅ | NHCH₂CH₂C₆H₅ | CH₂CHClC₆H₅ | 86.2 ± 8.1 |
| VS1-027 | SC₂H₅ | NHC₆H₄-mCl | CH₂CHClC₆H₅ | 99.0 ± 6.6 |
| VS1-028 | SCH₃ | NHCH₂CH₂C₆H₄-mCl | CH₂CHClC₆H₅ | 101.8 ± 10.1 |
| VS1-029 | H | NHCH₂C₆H₄-pF | CH₂CHClC₆H₄-pCl | 96.8 ± 4.5 |
| VS1-030 | SCH₃ | 4-MORPHOLINYL | CH₂CHClC₆H₅ | 98.6 ± 8.6 |
| VS1-031 | SCH₃ | NHC₃H₇ | CH₂CHClC₆H₅ | 103.1 ± 3.3 |
| VS1-032 | SCH₃ | N(C₂H₅)₂ | CH₂CHClC₆H₅ | 99.1 ± 3.7 |
| VS1-033 | SCH₃ | NH(CH₂)₂OC₂H₅ | CH₂CHClC₆H₅ | 108.1 ± 3.5 |
| VS1-034 | SCH₃ | 1-hexahydroazepinyl | CH₂CHClC₆H₅ | 105.0 ± 8.2 |
| VS1-035 | SCH₃ | NHCH₂C₆H₄-pCl | CH₂CHClC₆H₅ | 92.8 ± 23.1 |
| VS1-036 | SCH₃ | NHCH₂C₆H₄-oCl | CH₂CHClC₆H₅ | 102.3 ± 19.3 |

All the SPPs compounds reported in Table 1 showed acceptable or no cytotoxicity at 10 μM. Viability was comparable to that of the untreated control and of the reference compounds (HU 100 μM, HU 100 μM+ddI 2 μM and Imatinib 10 μM). At higher concentrations SPPs had problems of solubility or were toxic.

### Example 2

### Study of the Effects of the SPPs on Cell Hyperproliferation and on Apoptosis in Primary T Cells

We built up an experimental model using primary T cells to study the activity of the compounds on induced hyperproliferation of cells, which is a characteristic of both chronic viral diseases and cancer including HIV and leukemia. We used the system to distinguish between potent anti-proliferative compounds for the treatment of chronic viral diseases (anti-proliferative and not inducing apoptosis) and for the treatment of cancer (anti-proliferative and inducing apoptosis).

Unstimulated (quiescent/resting, day 0 to 5) primary human CD4 T-lymphocytes were obtained by magnetic bead separation from healthy, normal donors. After five days in culture, cells were activated with PHA (5 μg/ml) to induce hyperproliferation then, at day 7, IL-2 (20 U/ml) was added (stimulated, day 6 to 10). All available compounds were tested at a concentration of 10 μM. HU 100 μM and Imatinib 10 μM were also employed as comparators and the negative control was left untreated. At day 10, cells were harvested. Proliferation of T cells, cultured as described above, was studied by staining the cells with carboxyfluorescein diacetate-succinimidyl ester (CFSE) at day 0 (immediately after infection). For data analysis, the mitotic index (M) was used, calculating the sum of mitotic events at each proliferation cycle. To extract a relative number, M was normalized to the total number of cells acquired using the equation M=Σ[(Xₙ(T)−Xₙ(T)/2ⁿ)], that gives the number of mitotic events from the experimentally obtained values of the proportion of T cells under each division peak n (Xₙ) and the total T cells (T). To study the effect of the compounds on apoptosis T cells were cultured as described above and at day 10 cells were stained with Annexin V and 7-AAD, in order to determine the percentage of total apoptotic events.

### Effects of SPPs on Cellular Hyperproliferation and Apoptosis

TABLE 2
The effect of SPPs on cell proliferation was explored in comparison with HU and Imatinib
was used as a comparator. The mitotic index (M) was calculated and expressed as percentage
of the not treated control (indicated as 100%).

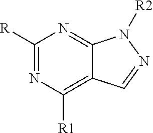

| | | | | mitotic |
|---|---|---|---|---|
| VS | R | R1 | R2 | index % |
| HU | — | — | — | 26.0 ± 2.0 |
| HU + ddI | — | — | — | 24.8 ± 7.5 |
| Imatinib | — | — | — | 37.6 ± 8.4 |
| VS1-001 | H | 1-hexahydroazepinyl | CHCHC₆H₅ | 52.0 ± 2.0 |
| VS1-002 | SC₂H₅ | NHC₄H₉ | CH₂CHClC₆H₅ | 25.2 ± 13.7 |
| VS1-003 | SCH₃ | NHCH₂C₆H₅ | CH₂CHClC₆H₅ | 43.0 ± 6.0 |
| VS1-004 | SCH₃ | NHCH₂CH₂C₆H₅ | CH₂CHClC₆H₅ | 23.1 ± 16.9 |
| VS1-005 | SCH₃ | NHCH₂CH₂C₆H₅ | CHCHC₆H₅ | 73.0 ± 5.0 |
| VS1-006 | H | NHC₄H₉ | CH₂CHBrC₆H₅ | 54.0 |
| VS1-007 | H | NHCYCLOHEXYL | CH₂CHBrC₆H₅ | 87.0 |
| VS1-008 | H | NHC₄H₉ | CH₂CHClC₆H₅ | 8.1 ± 1.8 |
| VS1-009 | H | 4-MORPHOLINYL | CHCHC₆H₅ | 88.4 ± 19.8 |
| VS1-010 | H | NHCH₂C₆H₅ | CH₂CHClC₆H₅ | 1.4 ± 0.6 |
| VS1-011 | SCH₃ | NHC₆H₄-mCl | CH₂CHClC₆H₅ | 0.4 ± 0.3 |
| VS1-012 | H | NHCYCLOHEXYL | CHCHC₆H₅ | 68.8 ± 10.7 |
| VS1-013 | H | NHCH₂C₆H₅ | CHCHC₆H₅ | 85.6 ± 3.6 |
| VS1-014 | H | NHCH₂CH₂C₆H₅ | CHCHC₆H₅ | 76.9 ± 2.7 |
| VS1-015 | NH(CH₂)₂OH | NHCH₂C₆H₅ | CHCHC₆H₅ | 55.4 ± 6.0 |
| VS1-016 | SCH₃ | NHC₆H₄-mCl | CHCHC₆H₅ | 71.2 ± 5.8 |
| VS1-017 | SCH₃ | NHCH₂C₆H₄-pF | CH₂CHClC₆H₅ | 34.5 ± 6.7 |
| VS1-018 | H | 1-hexahydroazepinyl | CH₂CHBrC₆H₅ | 42.4 ± 21.8 |
| VS1-019 | SCH₃ | NHCH₂C₆H₅ | CHCHC₆H₅ | 81.3 ± 18.9 |
| VS1-020 | SC₂H₅ | NHCH₂C₆H₅ | CH₂CHClC₆H₅ | 21.9 ± 13.0 |
| VS1-021 | H | NHC₄H₉ | CHCHC₆H₅ | 86.2 ± 13.7 |
| VS1-022 | SCH₃ | NHC₆H₄-mCl | CH₂CHClC₆H₄-pF | 10.4 ± 11.4 |
| VS1-023 | N(CH3)₂ | NHC₃H₇ | CH₂CHClC₆H₅ | 33.2 ± 6.5 |
| VS1-024 | SCH₃ | NHC₄H₉ | CH₂CHClC₆H₅ | 45.5 ± 10.9 |
| VS1-025 | SCH₂CH₂-4-MORPHOLINYL | NHC₃H₇ | CH₂CHClC₆H₅ | 61.6 ± 12.7 |
| VS1-026 | SC₂H₅ | NHCH₂CH₂C₆H₅ | CH₂CHClC₆H₅ | 24.9 ± 16.6 |
| VS1-027 | SC₂H₅ | NHC₆H₄-mCl | CH₂CHClC₆H₅ | 2.4 ± 1.5 |
| VS1-028 | SCH₃ | NHCH₂CH₂C₆H₄-mCl | CH₂CHClC₆H₅ | 15.2 ± 15.7 |
| VS1-029 | H | NHCH₂C₆H₄-pF | CH₂CHClC₆H₄-pCl | 4.4 ± 2.6 |
| VS1-030 | SCH₃ | 4-MORPHOLINYL | CH₂CHClC₆H₅ | 15.5 ± 4.8 |
| VS1-031 | SCH₃ | NHC₃H₇ | CH₂CHClC₆H₅ | 23.9 ± 18.4 |
| VS1-032 | SCH₃ | N(C₂H₅)₂ | CH₂CHClC₆H₅ | 22.0 ± 14.5 |
| VS1-033 | SCH₃ | NH(CH₂)₂OC₂H₅ | CH₂CHClC₆H₅ | 15.4 ± 6.7 |
| VS1-034 | SCH₃ | 1-hexahydroazepinyl | CH₂CHClC₆H₅ | 25.9 ± 12.9 |
| VS1-035 | SCH₃ | NHCH₂C₆H₄-pCl | CH₂CHClC₆H₅ | 33.8 ± 11.0 |
| VS1-036 | SCH₃ | NHCH₂C₆H₄-oCl | CH₂CHClC₆H₅ | 12.0 ± 6.4 |

HU 100 μM strongly decreased cell proliferation (by 74% compared to not treated control). As expected HU+ddI combination had the same anti-proliferative capacity. Imatinib was also effective in reducing cell proliferation (by 62% compared to not treated control). We therefore feel confident that our model is suitable to study the activity of compounds used as anticancer (anti-leukemia) agents. All the SPPs having a 2-chloro-2-phenylethyl side chain at N1 showed anti-proliferative activity. VS1-002 10 μM and VS1-004 were as effective as 100 μM HU in inhibiting cell proliferation and more effective than the same concentration of Imatinib. VS1-003 had a little inferior anti-proliferative capacity compared to HU, more similar to that of Imatinib. VS1-005, not bearing a 2-chloro-2-phenylethyl side chain at N1, only reduced proliferation by 27%. VS1-010, VS1-011 and VS1-029 were even more potent than HU since they inhibited cell proliferation by 99%, 100% and 95%, respectively, compared to an untreated control, though 10 times less concentrated.

We determined the capacity of the compounds to affect the percentages of apoptotic events after 10 days of treatment in primary CD4 T cells.

TABLE 3
Effects of the SPPs on apoptosis.

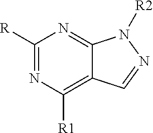

| VS | R | R1 | R2 | apoptosis % |
|---|---|---|---|---|
| HU | — | — | — | 95.9 ± 31.7 |
| HU + ddI | — | — | — | 140.2 ± 56.2 |
| Imatinib | — | — | — | 104.7 ± 18.6 |
| VS1-001 | H | 1-hexahydroazepinyl | CHCHC₆H₅ | 80.3 ± 19.2 |
| VS1-002 | SC₂H₅ | NHC₄H₉ | CH₂CHClC₆H₅ | 114.4 ± 51.7 |
| VS1-003 | SCH₃ | NHCH₂C₆H₅ | CH₂CHClC₆H₅ | 64.4 ± 15.1 |
| VS1-004 | SCH₃ | NHCH₂CH₂C₆H₅ | CH₂CHClC₆H₅ | 139.8 ± 64.7 |
| VS1-005 | SCH₃ | NHCH₂CH₂C₆H₅ | CHCHC₆H₅ | 51.9 ± 12.7 |
| VS1-006 | H | NHC₄H₉ | CH₂CHBrC₆H₅ | ND |
| VS1-007 | H | NHCYCLOHEXYL | CH₂CHBrC₆H₅ | ND |
| VS1-008 | H | NHC₄H₉ | CH₂CHClC₆H₅ | 115.8 ± 39.4 |
| VS1-009 | H | 4-MORPHOLINYL | CHCHC₆H₅ | 114.2 ± 21.3 |
| VS1-010 | H | NHCH₂C₆H₅ | CH₂CHClC₆H₅ | 149.8 ± 40.0 |
| VS1-011 | SCH₃ | NHC₆H₄-mCl | CH₂CHClC₆H₅ | 281.6 ± 145.0 |
| VS1-012 | H | NHCYCLOHEXYL | CHCHC₆H₅ | 46.3 ± 9.1 |
| VS1-013 | H | NHCH₂C₆H₅ | CHCHC₆H₅ | 121.3 ± 14.1 |
| VS1-014 | H | NHCH₂CH₂C₆H₅ | CHCHC₆H₅ | 135.2 ± 19.3 |
| VS1-015 | NH(CH₂)₂OH | NHCH₂C₆H₅ | CHCHC₆H₅ | 63.9 ± 9.2 |
| VS1-016 | SCH₃ | NHC₆H₄-mCl | CHCHC₆H₅ | 88.0 ± 12.7 |
| VS1-017 | SCH₃ | NHCH₂C₆H₄-pF | CH₂CHClC₆H₅ | 130.0 ± 19.3 |
| VS1-018 | H | 1-hexahydroazepinyl | CH₂CHBrC₆H₅ | 176.0 ± 22.5 |
| VS1-019 | SCH₃ | NHCH₂C₆H₅ | CHCHC₆H₅ | 94.0 ± 11.2 |
| VS1-020 | SC₂H₅ | NHCH₂C₆H₅ | CH₂CHClC₆H₅ | 174.7 ± 25.9 |
| VS1-021 | H | NHC₄H₉ | CHCHC₆H₅ | 108.0 ± 21.7 |
| VS1-022 | SCH₃ | NHC₆H₄-mCl | CH₂CHClC₆H₄-pF | 178.0 ± 25.7 |
| VS1-023 | N(CH3)₂ | NHC₃H₇ | CH₂CHClC₆H₅ | 116.0 ± 14.7 |
| VS1-024 | SCH₃ | NHC₄H₉ | CH₂CHClC₆H₅ | 126.7 ± 23.9 |
| VS1-025 | SCH₂CH₂-4-MORPHOLINYL | NHC₃H₇ | CH₂CHClC₆H₅ | 139.3 ± 20.8 |
| VS1-026 | SC₂H₅ | NHCH₂CH₂C₆H₅ | CH₂CHClC₆H₅ | 135.3 ± 15.7 |
| VS1-027 | SC₂H₅ | NHC₆H₄-mCl | CH₂CHClC₆H₅ | 90.1 ± 33.8 |
| VS1-028 | SCH₃ | NHCH₂CH₂C₆H₄-mCl | CH₂CHClC₆H₅ | 108.6 ± 30.8 |
| VS1-029 | H | NHCH₂C₆H₄-pF | CH₂CHClC₆H₄-pCl | 134.0 ± 33.1 |
| VS1-030 | SCH₃ | 4-MORPHOLINYL | CH₂CHClC₆H₅ | 174.4 ± 16.4 |
| VS1-031 | SCH₃ | NHC₃H₇ | CH₂CHClC₆H₅ | 114.4 ± 39.9 |
| VS1-032 | SCH₃ | N(C₂H₅)₂ | CH₂CHClC₆H₅ | 131.8 ± 58.6 |
| VS1-033 | SCH₃ | NH(CH₂)₂OC₂H₅ | CH₂CHClC₆H₅ | 138.6 ± 49.7 |
| VS1-034 | SCH₃ | 1-hexahydroazepinyl | CH₂CHClC₆H₅ | 115.1 ± 34.5 |
| VS1-035 | SCH₃ | NHCH₂C₆H₄-pCl | CH₂CHClC₆H₅ | 161.0 ± 13.2 |
| VS1-036 | SCH₃ | NHCH₂C₆H₄-oCl | CH₂CHClC₆H₅ | 139.3 ± 41.6 |

Both HU 100 μM and Imatinib 10 μM produced no significant increases in the percentage of apoptotic events, even though it is generally viewed as favorable for a cancer drug to induce apoptosis. VS1-002, VS1-003 and VS1-005 did not affect the percentage of apoptotic events compared to the control. VS1-004, VS1-010 and VS1-029 produced a slight increase in the percentage of apoptotic events, similar to the combination of HU 100 μM and ddI 2 μM. VS1-011 was strongly proapoptotic (+182% apoptotic events), compared to the control. VS1-018, VS1-020, VS1-022, VS1-030 and VS1-035 were also proapoptotic. Anti-proliferative compounds, which do not induce apoptosis are likely antiviral drugs and could be also used for the treatment of cancer, just as HU or Imatinib. However, more potent cancer drugs are both anti-proliferative and proapoptotic, such as VS1-030 and VS1-035.

### Example 3

### Study of the Antiviral Activity of the SPPs on HIV-1 Replication in Quiescent Cells

Our goal was to find a compound that, like HU, has good antiviral activity in a quiescent cell system.

Unstimulated (quiescent/resting, day 0 to 5) human CD4 T-lymphocytes, obtained by magnetic bead separation from healthy, normal donors, were infected with HIV-1 NL 4.3. After five days in culture, cells were stimulated with PHA (5 μg/ml) to induce proliferation, and then, at day 7, IL-2 (20 U/ml) was added (stimulated, day 6 to 10). All available compounds were tested at a concentration of 10 μM. HU 100 μM and HU 100 μM+ddI 2 μM were also employed as comparators and the negative control was left untreated. At day 10, cells and supernatants were harvested and HIV-1 p24 Ag was measured by ELISA. HIV replication was expressed as percentage p24 compared to the untreated control.

TABLE 4
Effects of SPPs on HIV replication in quiescent T cells.

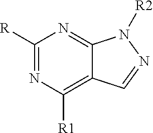

| | | | | HIV quiescent |
|---|---|---|---|---|
| VS | R | R1 | R2 | cells |
| HU | — | — | — | 13.0 ± 19.4 |
| HU + ddI | — | — | — | 3.8 ± 2.9 |
| Imatinib | — | — | — | 10.6 ± 8.2 |
| VS1-001 | H | 1-hexahydroazepinyl | CHCHC₆H₅ | 19.6 ± 20.3 |
| VS1-002 | SC₂H₅ | NHC₄H₉ | CH₂CHClC₆H₅ | 9.9 ± 9.4 |
| VS1-003 | SCH₃ | NHCH₂C₆H₅ | CH₂CHClC₆H₅ | 5.5 ± 8.9 |
| VS1-004 | SCH₃ | NHCH₂CH₂C₆H₅ | CH₂CHClC₆H₅ | 7.3 ± 11.4 |
| VS1-005 | SCH₃ | NHCH₂CH₂C₆H₅ | CHCHC₆H₅ | 2.6 ± 3.1 |
| VS1-006 | H | NHC₄H₉ | CH₂CHBrC₆H₅ | 17.8 ± 2.5 |
| VS1-007 | H | NHCYCLOHEXYL | CH₂CHBrC₆H₅ | 75.7 ± 18.8 |
| VS1-008 | H | NHC₄H₉ | CH₂CHClC₆H₅ | 11.2 ± 7.7 |
| VS1-009 | H | 4-MORPHOLINYL | CHCHC₆H₅ | 71.8 ± 42.9 |
| VS1-010 | H | NHCH₂C₆H₅ | CH₂CHClC₆H₅ | 10.6 ± 8.2 |
| VS1-011 | SCH₃ | NHC₆H₄-mCl | CH₂CHClC₆H₅ | 20.4 ± 4.0 |
| VS1-012 | H | NHCYCLOHEXYL | CHCHC₆H₅ | 10.9 ± 5.0 |
| VS1-013 | H | NHCH₂C₆H₅ | CHCHC₆H₅ | 63.3 ± 26.0 |
| VS1-014 | H | NHCH₂CH₂C₆H₅ | CHCHC₆H₅ | 74.3 ± 22.8 |
| VS1-015 | NH(CH₂)₂OH | NHCH₂C₆H₅ | CHCHC₆H₅ | 7.6 ± 2.8 |
| VS1-016 | SCH₃ | NHC₆H₄-mCl | CHCHC₆H₅ | 23.6 ± 22.1 |
| VS1-017 | SCH₃ | NHCH₂C₆H₄-pF | CH₂CHClC₆H₅ | 38.7 ± 44.5 |
| VS1-018 | H | 1-hexahydroazepinyl | CH₂CHBrC₆H₅ | 11.5 ± 3.2 |
| VS1-019 | SCH₃ | NHCH₂C₆H₅ | CHCHC₆H₅ | 46.7 ± 31.3 |
| VS1-020 | SC₂H₅ | NHCH₂C₆H₅ | CH₂CHClC₆H₅ | 9.2 ± 3.7 |
| VS1-021 | H | NHC₄H₉ | CHCHC₆H₅ | 115.4 ± 40.4 |
| VS1-022 | SCH₃ | NHC₆H₄-mCl | CH₂CHClC₆H₄-pF | 5.4 ± 3.1 |
| VS1-023 | N(CH3)₂ | NHC₃H₇ | CH₂CHClC₆H₅ | 8.7 ± 5.6 |
| VS1-024 | SCH₃ | NHC₄H₉ | CH₂CHClC₆H₅ | 24.3 ± 9.4 |
| VS1-025 | SCH₂CH₂-4-MORPHOLINYL | NHC₃H₇ | CH₂CHClC₆H₅ | 23.6 ± 14.5 |
| VS1-026 | SC₂H₅ | NHCH₂CH₂C₆H₅ | CH₂CHClC₆H₅ | 29.3 ± 6.9 |
| VS1-027 | SC₂H₅ | NHC₆H₄-mCl | CH₂CHClC₆H₅ | 2.7 ± 1.6 |
| VS1-028 | SCH₃ | NHCH₂CH₂C₆H₄-mCl | CH₂CHClC₆H₅ | 4.4 ± 2.9 |
| VS1-029 | H | NHCH₂C₆H₄-pF | CH₂CHClC₆H₄-pCl | 6.1 ± 3.9 |
| VS1-030 | SCH₃ | 4-MORPHOLINYL | CH₂CHClC₆H₅ | 17.4 ± 10.8 |
| VS1-031 | SCH₃ | NHC₃H₇ | CH₂CHClC₆H₅ | 19.1 ± 1.8 |
| VS1-032 | SCH₃ | N(C₂H₅)₂ | CH₂CHClC₆H₅ | 19.7 ± 7.2 |
| VS1-033 | SCH₃ | NH(CH₂)₂OC₂H₅ | CH₂CHClC₆H₅ | 15.0 ± 3.0 |
| VS1-034 | SCH₃ | 1-hexahydroazepinyl | CH₂CHClC₆H₅ | 22.2 ± 7.0 |
| VS1-035 | SCH₃ | NHCH₂C₆H₄-pCl | CH₂CHClC₆H₅ | 24.0 ± 20.0 |
| VS1-036 | SCH₃ | NHCH₂C₆H₄-oCl | CH₂CHClC₆H₅ | 14.5 ± 4.0 |

HU 100 μM strongly affected HIV replication (13.0±19.4% HIV p24 compared to untreated control). HU 100 μM+ddI 2 μM had increased antiviral potency. All the SPPs having a 2-chloro-2-phenylethyl side chain at N1 (R2 position) showed antiviral activity. 10 μM of VS1-002, VS1-003, VS1-004, VS1-010 and VS1-029 were as well strong HIV inhibitors as they reduced HIV-1 p24 production by 90%, 95%, 93%, 89% and 94% compared to untreated control, respectively. VS1-011 was a little less potent (80% inhibition of HIV replication). VS1-005, bearing a styryl group at N1, also showed a 97% inhibition of HIV replication.

### Example 4

### Study of the Antiviral Activity of the SPPs on HIV-1 Replication in Activated Cells

HU has little, if any anti-HIV activity in activated T cells because it is active against subunit R2 of ribonucleotide reductase, which is over-expressed upon cell cycle progression from G1 to S phase. A good candidate compound should also be active in activated T cells. The experimental setup described below allows us to detect direct antiviral activity of a compound. If a compound is not anti-proliferative but it inhibits HIV in activated T cells, the compound has a direct anti-HIV activity.

Human PBMC, obtained from healthy, normal donors, were stimulated for two days with PHA 5 μg/ml. Then cells were infected with HIV-1 NL 4.3, IL-2 was added (20 U/ml), cells were plated (10⁵ cells per well in 200 μl) and drug treatment started. 100 μM HU (comparator), 10 μM of Imatinib and of each SPP were tested in triplicates. The control was left untreated. After 7 days of incubation at 37° C. supernatants were harvested and HIV-1 p24 Ag was measured by ELISA.

TABLE 5
Effects of drugs on HIV replication in activated T cells.

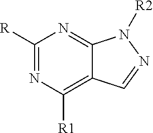

| | | | | HIV activated |
|---|---|---|---|---|
| VS | R | R1 | R2 | cells |
| HU | — | — | — | 82.6 ± 16.2 |
| HU + ddI | — | — | — | 1.9 ± 1.2 |
| Imatinib | — | — | — | 11.8 ± 5.6 |
| VS1-001 | H | 1-hexahydroazepinyl | CHCHC₆H₅ | 12.5 ± 2.3 |
| VS1-002 | SC₂H₅ | NHC₄H₉ | CH₂CHClC₆H₅ | 13.6 ± 13.0 |
| VS1-003 | SCH₃ | NHCH₂C₆H₅ | CH₂CHClC₆H₅ | 23.8 ± 7.6 |
| VS1-004 | SCH₃ | NHCH₂CH₂C₆H₅ | CH₂CHClC₆H₅ | 11.6 ± 11.7 |
| VS1-005 | SCH₃ | NHCH₂CH₂C₆H₅ | CHCHC₆H₅ | 10.3 ± 5.9 |
| VS1-006 | H | NHC₄H₉ | CH₂CHBrC₆H₅ | 100.5 ± 26.4 |
| VS1-007 | H | NHCYCLOHEXYL | CH₂CHBrC₆H₅ | 70.6 ± 24.3 |
| VS1-008 | H | NHC₄H₉ | CH₂CHClC₆H₅ | 11.8 ± 5.6 |
| VS1-009 | H | 4-MORPHOLINYL | CHCHC₆H₅ | 44.8 ± 14.9 |
| VS1-010 | H | NHCH₂C₆H₅ | CH₂CHClC₆H₅ | 11.8 ± 5.6 |
| VS1-011 | SCH₃ | NHC₆H₄-mCl | CH₂CHClC₆H₅ | 15.5 ± 2.6 |
| VS1-012 | H | NHCYCLOHEXYL | CHCHC₆H₅ | 18.6 ± 2.9 |
| VS1-013 | H | NHCH₂C₆H₅ | CHCHC₆H₅ | 116.4 ± 21.1 |
| VS1-014 | H | NHCH₂CH₂C₆H₅ | CHCHC₆H₅ | 92.4 ± 24.3 |
| VS1-015 | NH(CH₂)₂OH | NHCH₂C₆H₅ | CHCHC₆H₅ | 15.3 ± 2.4 |
| VS1-016 | SCH₃ | NHC₆H₄-mCl | CHCHC₆H₅ | 21.0 ± 6.3 |
| VS1-017 | SCH₃ | NHCH₂C₆H₄-pF | CH₂CHClC₆H₅ | 63.4 ± 31.2 |
| VS1-018 | H | 1-hexahydroazepinyl | CH₂CHBrC₆H₅ | 93.6 ± 28.3 |
| VS1-019 | SCH₃ | NHCH₂C₆H₅ | CHCHC₆H₅ | 89.3 ± 25.5 |
| VS1-020 | SC₂H₅ | NHCH₂C₆H₅ | CH₂CHClC₆H₅ | 26.3 ± 6.1 |
| VS1-021 | H | NHC₄H₉ | CHCHC₆H₅ | 97.2 ± 30.3 |
| VS1-022 | SCH₃ | NHC₆H₄-mCl | CH₂CHClC₆H₄-pF | 37.1 ± 19.8 |
| VS1-023 | N(CH3)₂ | NHC₃H₇ | CH₂CHClC₆H₅ | 36.6 ± 15.1 |
| VS1-024 | SCH₃ | NHC₄H₉ | CH₂CHClC₆H₅ | 10.1 ± 7.4 |
| VS1-025 | SCH₂CH₂-4-MORPHOLINYL | NHC₃H₇ | CH₂CHClC₆H₅ | 74.2 ± 31.7 |
| VS1-026 | SC₂H₅ | NHCH₂CH₂C₆H₅ | CH₂CHClC₆H₅ | 19.6 ± 12.6 |
| VS1-027 | SC₂H₅ | NHC₆H₄-mCl | CH₂CHClC₆H₅ | 12.0 ± 9.5 |
| VS1-028 | SCH₃ | NHCH₂CH₂C₆H₄-mCl | CH₂CHClC₆H₅ | 25.7 ± 19.8 |
| VS1-029 | H | NHCH₂C₆H₄-pF | CH₂CHClC₆H₄-pCl | 12.3 ± 8.8 |
| VS1-030 | SCH₃ | 4-MORPHOLINYL | CH₂CHClC₆H₅ | 34.5 ± 13.4 |
| VS1-031 | SCH₃ | NHC₃H₇ | CH₂CHClC₆H₅ | 31.6 ± 21.7 |
| VS1-032 | SCH₃ | N(C₂H₅)₂ | CH₂CHClC₆H₅ | 59.3 ± 8.1 |
| VS1-033 | SCH₃ | NH(CH₂)₂OC₂H₅ | CH₂CHClC₆H₅ | 57.1 ± 10.2 |
| VS1-034 | SCH₃ | 1-hexahydroazepinyl | CH₂CHClC₆H₅ | 65.0 ± 12.0 |
| VS1-035 | SCH₃ | NHCH₂C₆H₄-pCl | CH₂CHClC₆H₅ | 49.5 ± 8.1 |
| VS1-036 | SCH₃ | NHCH₂C₆H₄-oCl | CH₂CHClC₆H₅ | 22.1 ± 5.7 |

As expected, 100 μM HU was not effective in inhibiting HIV replication in activated T cells and it was necessary to add ddI 2 μM to achieve optimal antiviral activity. Conversely, all SPPs having a 2-chloro-2-phenylethyl side chain at N1 (R2 substitution) showed antiviral activity. 10 μM of SPPs had in many cases activity only little inferior to the HU/ddI combination. For example, VS1-002 and VS1-004 were strongly effective against HIV replication as % p24 was 13.6±13.0 and 11.6±11.7 respectively, compared to not treated control (they inhibited p24 production by 86% and 88% compared to untreated control, respectively). VS1-010, VS1-011 and VS1-029 were similarly effective as they reduced the % p24 to 11.8±5.6, 15.5±2.6 and 12.3±8.8, respectively, compared to not treated control (they reduced HIV replication by 88%, 85% and 88% compared to untreated control, respectively). Also Imatinib showed comparable antiviral capacity. VS1-005, bearing a styryl group at N1, showed the same antiviral activity. VS1-003 had slightly inferior anti-HIV capacity, % p24 was 23.8±7.6 compared to not treated control (it inhibited viral replication by 76%).

### Example 5

### Study of the Effects of Specific SPPs on Cell Cycle

A 48 well plate was coated with anti-CD3 antibody by dispensing 100 microliter of a 10 μg/ml antibody solution followed by overnight incubation at 4° C. Human peripheral blood mononuclear cells (PBMC), obtained from healthy, normal donors, were then plated (2×10⁶ cells per well in 1 ml) in complete culture medium supplemented with anti-CD28 antibody (2 μg/ml). Stimulated cells were cultured for 2 days then treated with HU, CsA, and VS1-002 to induce cell cycle arrest. After 24 hours cells were collected, stained with Ki-67 antibody and 7-AAD, and analyzed at the flow cytometer to detect the percentage of cells in G0, G1, S and G2/M phases. Results are shown in FIG. 6.

Cyclosporin, a known G0/G1 inhibitor, provoked a decrease in the percentage of cells in G1 phase in parallel with an increase in the percentage of cells in G0 phase, compared to the not treated control. VS1-002 and HU (a G1/S blocker) both decreased the percentage of cells in S phase with an increase in the percentage of cells in G1 phase of the cell cycle, compared to the not treated control. According to our results also SPPs only 10 μM like HU 100 μM limits cell cycle progression at the G1/S boundary.

### Antiviral-Hyperactivation Limiting Therapeutics (AV-HALTS)

In Table 6 there is a list of compounds endowed with both good (++, 50 to 70% activity) or even excellent (+++, greater than 70% activity) anti-proliferative and antiviral capacity. This represents an improvement over the combination of the anti-proliferative drug HU and the antiviral drug ddI.

TABLE 6
AV-HALTS

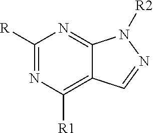

| | | | | Anti- | Antiviral | Antiviral |
|---|---|---|---|---|---|---|
| | | | | proliferative | activity in | activity in |
| VS | R | R1 | R2 | capacity | activated cells | quiescent cells |
| HU + ddI | — | — | — | +++ | +++ | +++ |
| VS1-002 | SC₂H₅ | NHC₄H₉ | CH₂CHClC₆H₅ | +++ | +++ | +++ |
| VS1-003 | SCH₃ | NHCH₂C₆H₅ | CH₂CHClC₆H₅ | ++ | +++ | +++ |
| VS1-004 | SCH₃ | NHCH₂CH₂C₆H₅ | CH₂CHClC₆H₅ | +++ | +++ | +++ |
| VS1-008 | H | NHC₄H₉ | CH₂CHClC₆H₅ | +++ | +++ | +++ |
| VS1-024 | SCH₃ | NHC₄H₉ | CH₂CHClC₆H₅ | ++ | +++ | +++ |
| VS1-026 | SC₂H₅ | NHCH₂CH₂C₆H₅ | CH₂CHClC₆H₅ | +++ | +++ | ++ |
| VS1-027 | SC₂H₅ | NHC₆H₄-mCl | CH₂CHClC₆H₅ | +++ | +++ | +++ |
| VS1-028 | SCH₃ | NHCH₂CH₂C₆H₄-mCl | CH₂CHClC₆H₅ | +++ | +++ | +++ |
| VS1-029 | H | NHCH₂C₆H₄-pF | CH₂CHClC₆H₄-pCl | +++ | +++ | +++ |
| VS1-031 | SCH₃ | NHC₃H₇ | CH₂CHClC₆H₅ | +++ | ++ | +++ |
| VS1-036 | SCH₃ | NHCH₂C₆H₄-oCl | CH₂CHClC₆H₅ | +++ | +++ | +++ |

1. A method of inhibiting cellular proliferation and/or inhibiting viral activity and/or inhibiting tumor growth by administering an effective amount of 4-substituted derivatives of pyrazolo[3,4-d]pyrimidine (SPPs).

2. The method of claim 1 wherein the 4-substituted derivatives of pyrazolo[3,4-d]pyrimidine (SPPs) that inhibit cellular proliferation have the formula 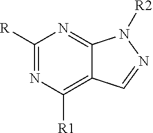
wherein:
R=SC2H5, SCH3, H, N(CH3)2
R1=NHC4H9, NHCH2CH2C6H5, NHCH2C6H5, NHC6H4-mCl, NHCH2C6H4-pF, NHC3H7, NHCH2CH2C6H4-mCl, 4-MORPHOLINYL, N(C2H5)2, NH(CH2)2OC2H5, 1-HEXAHYDROAZEPINYL, NHCH2C6H4-pCl, NHCH2C6H4-oCl
R2=CH2CHClC6H5, CH2CHClC6H4-pF, CH2CHClC6H4-pCl.

3. The method of claim 1 wherein the 4-substituted derivatives of pyrazolo[3,4-d]pyrimidine (SPPs) that induce apoptosis have the formula 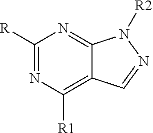
wherein:
R=SC2H5, SCH3, H, SCH2CH2-4-MORPHOLINYL
R1=NHCH2CH2C6H5, NHCH2C6H5, NHC6H4-mCl, 1-HEXAHYDROAZEPINYL, NHC3H7, 4-MORPHOLINYL, NHCH2C6H4-pCl
R2=═CH2CHClC6H5, CH2CHBrC6H5, CH2CHClC6H4-pF, CH2CHClC6H4-pCl.

4. The method of claim 1 wherein the 4-substituted derivatives of pyrazolo[3,4-d]pyrimidine (SPPs) that inhibit viral replication have the formula 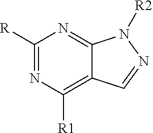
Wherein:
R=H, SCH3, NH(CH2)2OH
R1=1-HEXAHYDROAZEPINYL, NHCH2CH2C6H5,4-MORPHOLINYL, NHCYCLOHEXYL, NHCH2C6H5, NHC6H4-mCl
R2=CHCHC6H5.

5. The method of claim 1 wherein the 4-substituted derivatives of pyrazolo[3,4-d]pyrimidine (SPPs) that inhibit cell proliferation and viral replication have the formula 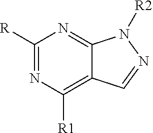
wherein:
R=SC2H5, SCH3, H, N(CH3)2
R1=NHC4H9, NHCH2CH2C6H5, NHCH2C6H5, NHC6H4-mCl, NHCH2C6H4-pF, NHC3H7, NHCH2CH2C6H4-mCl, 4-MORPHOLINYL, NHCH2C6H4-oCl
R2=CH2CHClC6H5, CH2CHClC6H4-pF, CH2CHClC6H4-pCl.

6. Use of any one of the compounds of claim 2 or 3 for anti-tumor and anti-leukemia therapy.

7. Use of any one of the compounds of claim 4 to inhibit viral replication.

8. Use of any of the compounds of claim 5 to limit hyperactivation or hyperproliferation of the immune system and limit viral replication.

9. A pharmaceutical composition comprising a pyrazolo[3,4-d]pyrimidine derivative of claim 1 with a 2-chloro-2-phenylethyl or a 2-chloro-2-(4-fluoro-phenyl)ethyl or a 2-chloro-2-(4-chloro-phenyl)ethyl side chain at N1 (substituent R2) to inhibit cell proliferation and/or viral replication.

10. A pharmaceutical composition comprising a pyrazolo[3,4-d]pyrimidine derivative of claim 1 with a SC2H5 side chain at C6 (substituent R) to inhibit cell proliferation and/or viral replication.

11. A pharmaceutical composition comprising a pyrazolo[3,4-d]pyrimidine derivative of claim 1 with a styryl side chain at N1 (substituent R2) to inhibit viral replication.

12. Use of any compounds of claims 1-5 to limit cell cycle progression beyond G1/S checkpoint.

13. The method of claim 5, wherein the compounds inhibiting both T cell proliferation and viral replication, are selected from the group consisting of VS1-002, VS1-003, VS1-004, VS1-008, VS1-024, VS1-026, VS1-027, VS1-028, VS1-029, VS1-031, VS1-036.

14. A method of claim 4, wherein the compounds inhibiting viral replication are selected from the group consisting of VS1-001, VS1-005, VS1-012, VS1-015, VS1-016.

15. A method of claim 6, wherein the compounds inhibiting leukemia or tumor growth are selected from the group consisting of VS1-010, VS1-011, VS1-020, VS1-022, VS1-023, VS1-030, VS1-032, VS1-033, VS1-034, VS1-035.
